(19) United States
(12) Patent Application Publication
(10) Pub. No.: US 2026/0306579 A1
(43) Pub. Date: Oct. 8, 2026

(54) COMPOSITIONS AND METHODS FOR TREATMENT OF SEPTIC SHOCK

(71) Applicant: PEROXITECH THERAPEUTICS, INC., Philadelphia, PA (US)

(72) Inventor: Thomas Han, Philadelphia, PA (US)

(21) Appl. No.: 19/481,498

(22) PCT Filed: May 3, 2024
(86) PCT No.: PCT/US2024/027825

§ 371 Date: Nov. 4, 2025

Related U.S. Application Data
(60) Provisional application No. 63/500,286, filed on May 5, 2023.

(51) Int. Cl. A61K 38/08; A61K 38/10; A61P 9/02; A61P 11/00; A61P 43/00

(52) U.S. Cl. CPC A61K 38/08; A61K 38/10; A61P 9/02; A61P 11/00; A61P 43/00

(57) ABSTRACT

The present application provides for polypeptides for the treatment of septic shock and methods of use thereof.

### CROSS REFERENCE TO RELATED APPLICATIONS

This application claims the benefit of and priority to U.S. Provisional Application No. 63/500,286, filed May 5, 2023, which is hereby incorporated by reference in its entirety for all purposes.

### SEQUENCE LISTING

The instant application contains a Sequence Listing which is hereby incorporated by reference in its entirety. Said XML copy, created on May 3, 2024, is named PEX-002WO_SL.xml, and is 32,768 bytes in size.

### BACKGROUND

Septic shock is the last and most severe stage of sepsis, where dangerous low and severe drops in blood pressure and/or increased lactate levels and cannot be adequately treated by the administration of fluids alone. Even with the treatment of vasopressors to maintain blood pressure, the mortality rate for septic shock is high, ranging from 25-50% (see, e.g., Kumar et al, eds., Robbins Basic Pathology (8^{th} ed.), Saunders, Elsevier pp. 102-3 (2007); Singer et al., “The Third International Consensus Definitions for Sepsis and Septic Shock (Sepsis-3),” JAMA 315(8):801-10 (2016), which are each incorporated by reference in their entireties). Thus, there is a current need for compositions and methods that can treat septic shock. The present embodiments fulfill these needs as well as others.

### SUMMARY

Methods of treating septic shock in a subject in need thereof are provided herein, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
   wherein: X¹ is E or is absent; X² is L or is absent; X³ is Q or is absent; X⁴ is A, T, or is absent; X⁵ is T, E, or is absent; X⁶ is H or Y; X⁷ is D or E; X⁸ is F or I; and X⁹ is R or K; or an amino acid sequence selected from the group consisting of SEQ ID NOs: 10, 11, 12, 13, 14, 15, 17, 18, 19, 20, 21, 22, 24, 25, 26, 27, 28, 29, or 30; and a pharmaceutically acceptable carrier. In some embodiments, the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises SEQ ID NO: 2. In some embodiments, the polypeptide consists of SEQ ID NO: 2. In some embodiments, the polypeptide comprises SEQ ID NO: 3. In some embodiments, the polypeptide consists of SEQ ID NO: 3. In some embodiments, the polypeptide comprises SEQ ID NO: 4. In some embodiments, the polypeptide consists of SEQ ID NO: 4. In some embodiments, the pharmaceutical composition comprises a liposome encapsulated polypeptide. In some embodiments, the pharmaceutically acceptable carrier is any pharmaceutically acceptable carrier disclosed herein. In some embodiments, the pharmaceutical composition is administered to the subject by any administration method disclosed herein. In some embodiments, the pharmaceutical composition is administered to the subject by aerosol inhalation, intratracheal injection, or intravenous injection. In some embodiments, the subject in need thereof has been diagnosed with sepsis and has low blood pressure that is not alleviated by the administration of intravenous fluids alone. In some embodiments, the subject in need thereof has a mean arterial pressure (MAP) less than about 65 mm Hg. In some embodiments, the subject in need thereof has been diagnosed with sepsis and has a decrease in mean arterial pressure or systolic blood pressure of about 44 mm Hg or more. In some embodiments, the subject in need thereof has blood lactate levels that are greater than about 4 mmol. In some embodiments, subject in need thereof has persistent signs of multiple organ damage or failure. In some embodiments, the subject's blood oxygen level does not decrease after administration of the pharmaceutical composition. In some embodiments, the subject's blood oxygen level does not decrease more than 50% after administration of the pharmaceutical composition. In some embodiments, the subject's blood oxygen level does not decrease more than 65% after administration of the pharmaceutical composition. In some embodiments, the subject's blood oxygen level increases after administration of the pharmaceutical composition. In some embodiments, the subject maintains a normal blood oxygen level after administration of the pharmaceutical composition. In some embodiments, the subject in need thereof is not administered a vasopressor or the subject has been previously administered a lower amount of a vasopressor, is concurrently administered a lower amount of a vasopressor, or will subsequently be administered a lower amount of a vasopressor than the amount of a vasopressor administered in the absence of the pharmaceutical composition. In some embodiments, the subject in need thereof has been diagnosed with septic shock.

Methods of treating subject with a significant drop in mean arterial pressure are also provided for herein, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
   wherein: X¹ is E or is absent; X² is L or is absent; X³ is Q or is absent; X⁴ is A, T, or is absent; X⁵ is T, E, or is absent; X⁶ is H or Y; X⁷ is D or E; X⁸ is F or I; and X⁹ is R or K; or an amino acid sequence selected from the group consisting of SEQ ID NOs: 10, 11, 12, 13, 14, 15, 17, 18, 19, 20, 21, 22, 24, 25, 26, 27, 28, 29, or 30; and a pharmaceutically acceptable carrier. In some embodiments, the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises SEQ ID NO: 2. In some embodiments, the polypeptide consists of SEQ ID NO: 2. In some embodiments, the polypeptide comprises SEQ ID NO: 3. In some embodiments, the polypeptide consists of SEQ ID NO: 3. In some embodiments, the polypeptide comprises SEQ ID NO: 4. In some embodiments, the polypeptide consists of SEQ ID NO: 4. In some embodiments, the pharmaceutical composition comprises a liposome encapsulated polypeptide. In some embodiments, the pharmaceutically acceptable carrier is any pharmaceutically acceptable carrier disclosed herein. In some embodiments, the pharmaceutical composition is administered to the subject by any administration method disclosed herein. In some embodiments, the pharmaceutical composition is administered to the subject by aerosol inhalation, intratracheal injection, or intravenous injection. In some embodiments, the subject has a decrease in mean arterial pressure or systolic blood pressure of about 44 mm Hg or more and has been diagnosed with sepsis. In some embodiments, the subject has been diagnosed with sepsis and has low blood pressure that is not alleviated by the administration of intravenous fluids alone. In some embodiments, the subject has a mean arterial pressure (MAP) less than about 65 mm Hg. In some embodiments, the subject in need thereof has blood lactate levels that are greater than about 4 mmol. In some embodiments, the subject in need thereof has persistent signs of multiple organ damage or failure. In some embodiments, the subject's blood oxygen level does not decrease after administration of the pharmaceutical composition. In some embodiments, the subject's blood oxygen level does not decrease more than 50% after administration of the pharmaceutical composition. In some embodiments, the subject's blood oxygen level does not decrease more than 65% after administration of the pharmaceutical composition. In some embodiments, the subject's blood oxygen level increases after administration of the pharmaceutical composition. In some embodiments, the subject maintains a normal blood oxygen level after administration of the pharmaceutical composition. In some embodiments, the subject in need thereof is not administered a vasopressor or the subject has been previously administered, is concurrently administered, or will subsequently be administered a lower amount of a vasopressor than the amount of a vasopressor administered in the absence of the pharmaceutical composition. In some embodiments, the subject in need thereof has been diagnosed with septic shock.

Methods of preventing heart damage in a subject with diagnosed with septic shock are also provided for herein, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
   wherein: X¹ is E or is absent; X² is L or is absent; X³ is Q or is absent; X⁴ is A, T, or is absent; X⁵ is T, E, or is absent; X⁶ is H or Y; X⁷ is D or E; X⁸ is F or I; and X⁹ is R or K; or an amino acid sequence selected from the group consisting of SEQ ID NOs: 10, 11, 12, 13, 14, 15, 17, 18, 19, 20, 21, 22, 24, 25, 26, 27, 28, 29, or 30; and a pharmaceutically acceptable carrier, as compared to a subject not treated with the pharmaceutical composition. In some embodiments, the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises SEQ ID NO: 2. In some embodiments, the polypeptide consists of SEQ ID NO: 2. In some embodiments, the polypeptide comprises SEQ ID NO: 3. In some embodiments, the polypeptide consists of SEQ ID NO: 3. In some embodiments, the polypeptide comprises SEQ ID NO: 4. In some embodiments, the polypeptide consists of SEQ ID NO: 4. In some embodiments, the pharmaceutical composition comprises a liposome encapsulated polypeptide. In some embodiments, the pharmaceutically acceptable carrier is any pharmaceutically acceptable carrier disclosed herein. In some embodiments, the pharmaceutical composition is administered to the subject by any administration method disclosed herein. In some embodiments, the pharmaceutical composition is administered to the subject by aerosol inhalation, intratracheal injection, or intravenous injection. In some embodiments, the subject in need thereof has low blood pressure that is not alleviated by the administration of intravenous fluids alone. In some embodiments, the subject in need thereof has a mean arterial pressure (MAP) less than about 65 mm Hg. In some embodiments, the subject in need thereof has a decrease in mean arterial pressure or systolic blood pressure of about 44 mm Hg or more. In some embodiments, the subject in need thereof has blood lactate levels that are greater than about 4 mmol. In some embodiments, subject in need thereof has persistent signs of multiple organ damage or failure. In some embodiments, subject's blood oxygen level does not decrease after administration of the pharmaceutical composition. In some embodiments, the subject's blood oxygen level does not decrease more than 50% after administration of the pharmaceutical composition. In some embodiments, the subject's blood oxygen level does not decrease more than 65% after administration of the pharmaceutical composition. In some embodiments, the subject's blood oxygen level increases after administration of the pharmaceutical composition. In some embodiments, the subject maintains a normal blood oxygen level after administration of the pharmaceutical composition. In some embodiments, the subject in need thereof has a troponin level that that does not increase after administration of the pharmaceutical composition In some embodiments, the subject in need thereof maintains normal troponin levels after administration of the pharmaceutical composition. In some embodiments, the subject in need thereof is not administered a vasopressor or the subject has been previously administered, is concurrently administered, or will subsequently be administered a lower amount of a vasopressor than the amount of a vasopressor administered in the absence of the pharmaceutical composition.

Methods of increasing survival of a subject with diagnosed with septic shock, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
   wherein: X¹ is E or is absent; X² is L or is absent; X³ is Q or is absent; X⁴ is A, T, or is absent; X⁵ is T, E, or is absent; X⁶ is H or Y; X⁷ is D or E; X⁸ is F or I; and X⁹ is R or K; or an amino acid sequence selected from the group consisting of SEQ ID NOs: 10, 11, 12, 13, 14, 15, 17, 18, 19, 20, 21, 22, 24, 25, 26, 27, 28, 29, or 30; and a pharmaceutically acceptable carrier, as compared to a subject not treated with the pharmaceutical composition. In some embodiments, the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises SEQ ID NO: 2. In some embodiments, polypeptide consists of SEQ ID NO: 2. In some embodiments, polypeptide comprises SEQ ID NO: 3. In some embodiments, the polypeptide consists of SEQ ID NO: 3. In some embodiments, the polypeptide comprises SEQ ID NO: 4. In some embodiments, the polypeptide consists of SEQ ID NO: 4. In some embodiments, the pharmaceutical composition comprises a liposome encapsulated polypeptide. In some embodiments, the pharmaceutically acceptable carrier is any pharmaceutically acceptable carrier disclosed herein. In some embodiments, the pharmaceutical composition is administered to the subject by any administration method disclosed herein. In some embodiments, the pharmaceutical composition is administered to the subject by aerosol inhalation, intratracheal injection, or intravenous injection. In some embodiments, the subject in need thereof has been diagnosed with sepsis and has low blood pressure that is not alleviated by the administration of intravenous fluids alone. In some embodiments, the subject in need thereof has a mean arterial pressure (MAP) less than about 65 mm Hg. In some embodiments, the subject in need thereof has a decrease in mean arterial pressure or systolic blood pressure of about 44 mm Hg or more. In some embodiments, the subject in need thereof has blood lactate levels that are greater than about 4 mmol. In some embodiments, the subject in need thereof has persistent signs of multiple organ damage or failure. In some embodiments, the subject's blood oxygen level does not decrease after administration of the pharmaceutical composition. In some embodiments, the subject's blood oxygen level does not decrease more than 50% after administration of the pharmaceutical composition. In some embodiments, the subject's blood oxygen level does not decrease more than 65% after administration of the pharmaceutical composition. In some embodiments, the subject's blood oxygen level increases after administration of the pharmaceutical composition. In some embodiments, the subject maintains a normal blood oxygen level after administration of the pharmaceutical composition. In some embodiments, the subject in need thereof is not administered a vasopressor or the subject has been previously administered, is concurrently administered, or will subsequently be administered a lower amount of a vasopressor than the amount of a vasopressor administered in the absence of the pharmaceutical composition

### BRIEF DESCRIPTION OF THE DRAWINGS

FIG. 1 illustrates a measurement of troponin I levels in subjects over time. The negative control and subjects treated with PIP-2 treated showed no increase in troponin protein levels in the blood, while the subjects were not treated with PIP-2 showed an increase in troponin protein levels starting between 1 and 4 hours.

FIG. 2 illustrates a measurement of c-reactive protein (CRP) levels in subjects over time. The negative control and subjects treated with PIP-2 treated showed no increase in CRP protein levels in the blood, while the subjects not treated with PIP-2 showed an increase in CRP protein levels starting between 1 and 4 hours.

FIG. 3 shows the mean use of vasopressors per animal for negative control, PIP-2 treated, and subjects not treated with PIP-2. The PIP-2 treated subjects required about 66% less vasopressor treatment to maintain appropriate MAP levels.

FIGS. 4A and 4B depict the effect of PIP-2 on mean arterial pressure (MAP). FIG. 4A shows a bar graph of the percent change in mean arterial pressure (MAP) vs. baseline for pigs treated with LPS and LPS+PIP-2. FIG. 4B shows a graph of the MAP values for control−, LPS−, and LPS+PIP-2-treated pigs over 8 hours.

FIG. 5 shows a survival curve for pigs treated with LPS and LPS+PIP-2. Pigs were treated with LPS delivered by IV infusion over a 1 hour period. In addition to LPS, pigs also received either PIP-2 in liposomes (n=12) or liposomes alone (n=6) as a slow bolus injection IV at the end of the LPS infusion. Death of pigs due to sacrifice for humane considerations was recorded at hourly intervals. All surviving pigs were sacrificed at 8 hours after the start of LPS infusion. The addition of PIP-2 led to an increase in survival as compared to the control.

FIG. 6 illustrates that PIP-2 treatment resulted in a 50% decrease in pig mortality by humane sacrifice and a 67% decrease in the incidence of moderate to severe ARDS. FIG. 6 illustrates the severity of acute lung injury as indicated by arterial blood PO₂ (aPO₂) values during ventilation with 100% O₂ (FIO₂) for 3 groups of pigs (Group 1: liposomes alone, no LPS; Group 2: LPS+liposomes; and Group 3: LPS+PIP-2 in liposomes) measured at 8 hours after the start of LPS infusion. The percentage of pigs within each group that fell within the range for normal, mild, moderate, or severe ARDS as defined by aPO₂/FIO₂ (Horowitz index) is shown for those pigs that survived for 8 hours. The percentage of pigs that were euthanized for humane considerations prior to the 8 hour end of the study is shown. Total pigs were: 2 for control, 6 for LPS and 12 for LPS+PIP-2.

FIG. 7 illustrates that PIP-2 led to a statistically significant lesser decrease in lung compliance in subject treated with LPS to induce septic shock. Specifically, FIG. 7 illustrates static lung compliance for pigs in group 2 (LPS treated) and group 3 (LPS treated+PIP-2 treated). Static lung compliance in mechanically ventilated pigs was measured at hourly intervals between zero time and 8 hours after the start of LPS infusion. The points were connected by a continuous line. Each line represents one pig. Early termination of a line indicates euthanasia of the pig for humane considerations. The heavy-weighted line in each graph represents the mean slope for each set of lines. The slope indicates the rate of compliance decrease for each group of pigs and was −1.35+0.36 ml/cm H₂O for the LPS group and −0.56+0.41 L/cm H₂O for the LPS+PIP-2 pigs. On comparison of the 2 groups by t test for N=16, P<0.001.

### DETAILED DESCRIPTION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

The terms “substituting,” “substituted,” “mutating,” or “mutated” as used herein refer to altering, deleting, or inserting one or more amino acids or nucleotides in a polypeptide or polynucleotide sequence to generate a variant of that sequence.

The terms “polynucleotide” or “nucleic acid molecule” means a molecule comprising a chain of nucleotides covalently linked by a sugar-phosphate backbone or other equivalent covalent chemistry. Double and single-stranded DNAs and RNAs are typical example of polynucleotides.

The term “polypeptide” or “protein” means a molecule that comprises at least two amino acid resides linked by a peptide bond to form a polypeptide. In some embodiments, the term “peptide” can also be used.

The term “sepsis” as used herein means a potentially life-threatening condition caused by the body's response to an infection. In some embodiments, sepsis can lead to the failure of one or more organs. Without wishing to be bound by theory, sepsis is categorized into multiple stages, which increased in severity. The term “septic shock” as used here means the last and most severe stage of sepsis, usually defined by sepsis with persistent signs of organ damage, low blood pressure and, in some instance, lactate levels that measure greater than 2 mmol or 4 mmol that is not alleviated by the administration of intravenous fluids and/or where vasopressors are required to maintain mean arterial pressure (MAP) of greater than or equal to 65 mm Hg.

### Compositions

A composition comprising a polypeptide is provided, the polypeptide having the formula of:
X¹X²X³X⁴X⁵LX⁶X⁷X⁸X⁹HQIL (SEQ ID NO: 1)
   wherein:
X¹ is E or is absent;
X² is L or is absent;
X³ is Q or is absent;
X⁴ is A, T, or is absent;
X⁵ is T, E, or is absent;
X⁶ is H or Y;
X⁷ is D or E;
X⁸ is F or n; and
X⁹ is R or K; or
   a polypeptide comprising an amino acid sequence selected from the group consisting of SEQ ID NOs: 10, 11, 12, 13, 14, 15, 17, 18, 19, 20, 21, 22, 24, 25, 26, 27, 28, 29, or 30.

In some embodiments, the polypeptide is a polypeptide listed in Table 1.

TABLE 1

| SEQ ID NO: | SEQUENCE |
|---|---|
| 2 | LHDFRHQIL |
| 3 | LYEIKHQIL |
| 4 | LYDIRHQIL |
| 5 | DEELQATLHDFRHQIL |
| 6 | DEELQTELYEIKHQIL |
| 7 | ELQTELYEIKHQIL |
| 8 | QTELYEIKHQIL |
| 9 | ELYEIKHQIL |
| 10 | YEIKHQIL |
| 11 | IKHQIL |
| 12 | DEELQTELYEIKHQ |
| 13 | DEELQTELYEIK |
| 14 | DEELQTELYEI |
| 15 | DEELQTEL |
| 16 | TLHDFRHQIL |
| 17 | TLHDFRHQI |
| 18 | LHDFRHQI |
| 19 | ELYEIKHQI |
| 20 | LYEIKHQI |
| 21 | LKIEYHQIL |
| 22 | LRFDHHQIL |
| 23 | LHEFKHQIL |
| 24 | LFKLEHQIL |
| 25 | LHDFRDQIL |
| 26 | LHDFRPQIL |
| 27 | LHDFRHNIL |
| 28 | LHDFRHIIL |
| 29 | LHDFRHQLL |
| 30 | LHDFRHQTL |

In some embodiments, the polypeptide is at least 85%, 90%, 91%, 92%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

As used herein, the term “PIP-2” is the polypeptide having the sequence of SEQ ID NO: 2. As used herein, the term “PIP-4” is the polypeptide having the sequence of SEQ ID NO: 3

As used herein, the term “PIP-5” is the polypeptide having the sequence of SEQ ID NO: 4. In some embodiments, the polypeptide comprises SEQ ID NOs: 2, 3, or 4. In some embodiments, the polypeptide consists of SEQ ID NOs: 2, 3, or 4.

### Pharmaceutical Compositions

In another aspect, the present embodiments provide compositions, e.g., pharmaceutically acceptable compositions, which include a polypeptide as provided for herein which can be, for example, be formulated together with one or more excipients. In some embodiments, suitable excipients include, but are not limited to purified water, calcium carbonate, calcium phosphate, various sugars, starches, cellulose derivatives, gelatin, polymers such as polyethylene glycols, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof.

In some embodiments, the pharmaceutical composition further comprises a pharmaceutically acceptable carrier. In some embodiments, suitable pharmaceutically acceptable carries include, but are not limited to, water, silicone, waxes, petroleum jelly, polyethylene glycol, propylene glycol, liposomes, a lipid such as cholesterol, cationic lipids such as 1,2,-dioleoyl-3-trimethylammonium propane (DOTAP), 1,2,-dioleoyl-sn-glycero-3-phosphochiline (DOPC), and 1,2-dioleoyl-sn-glycero-3-phosphoethanolamine (DOPE), sugars such as mannitol and lactose, and other materials depending on the specific type of formulation used. In some embodiments, suitable pharmaceutically acceptable carries include, but are not limited to, nanoparticles such as gold or metallic nanoparticles.

In some embodiments, the polypeptide is encapsulated in or formulated with one or more lipids and liposomes. In some embodiments, the lipids and liposomes comprises a cationic lipid, such as, but not limited to, 1,2-Dioleoyl-3-Trimethylammonium-Propane (DOTAP), 1,2,-dioleoyl-sn-glycero-3-phosphochiline (DOPC), 1,2-dioleoyl-sn-glycero-3-phosphoethanolamine (DOPE), N-[1-(2,3-dioleyloxy)propyl]-N,N,N-trimethylammonium chloride (DOTMA), 5-carboxyspermylglycinedioctadecylamide (DOGS), 2,3-dioleyloxy-N-[2(spermine-carboxamido)ethyl]-N,N-dimethyl-1-propanamin-ium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), or C12-200. In some embodiments, the lipids and liposomes comprise, but are not limited to, dipalmitoyl phosphatidylcholine (DPPC), egg PC, phosphatidylglycerol (PG), egg PC, or cholesterol. In some embodiments, the lipids and liposomes comprise dipalmitoyl phosphatidylcholine (DPPC), egg PC, phosphatidylglycerol (PG), and cholesterol in the molar ratio 0.5, 0.25, 0.10, 0.15.

In some embodiments, the pharmaceutical composition comprises an encapsulated polypeptide, such as the polypeptides as provided for herein.

In some embodiments, the pharmaceutically acceptable carrier can be suitable for intravenous, intramuscular, intratracheal subcutaneous, parenteral, rectal, local, topical, spinal or epidermal administration (e.g. by injection or infusion). In some embodiments, the pharmaceutically acceptable carrier can be suitable for aerosol inhalation.

The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments the mode of administration is parenteral (e.g., intravenous, subcutaneous, intraperitoneal, intradermal, intramuscular, intravesicular). In some embodiments, the composition is administered by intravenous infusion or injection. In some embodiments, the composition is administered by intramuscular or subcutaneous injection. In some embodiments, the composition is administered by enteral, sublingual, inhalation, or intranasal. In some embodiments, the composition is administered locally, e.g., by injection, or topical application, to a target site. For example, the pharmaceutical compositions can be lyophilized and reconstituted for use prior to administration to the patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Compositions, such as pharmaceutical compositions, typically are sterile and stable under the conditions of manufacture and storage. The composition can be formulated as a solution, microemulsion, dispersion, liposome, or other ordered structure suitable for a high concentration of the active ingredient. Sterile injectable solutions can be prepared by incorporating the active compound (i.e., therapeutic molecule, nucleic acid molecule, cell, polypeptide, vector, etc.) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the pharmaceutical composition can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compositions as provided for herein by other than parenteral administration, it may be necessary to coat the compositions with, or co-administer the compositions with, a material to prevent its inactivation. The compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks, or, in some embodiments, the dosing schedule can be, once every month, every 2 months, every 3 months, or every 6 months. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of the compositions, vectors, cells, polypeptides, or nucleic acid molecules encoding the same. A “therapeutically effective amount” refers to an amount effective, at dosages and for periods of time necessary, to achieve the desired therapeutic result. A therapeutically effective amount of an active ingredient or molecule may vary according to factors such as the disease state, age, sex, and weight of the individual, and the ability of the therapeutic compound to elicit a desired response in the individual. A “therapeutically effective dosage” can, for example, inhibit a measurable parameter, e.g., tumor growth, by at least about 20%, by at least about 40%, by at least about 60%, and by at least about 80% relative to untreated subjects. The ability of a compound to inhibit a measurable parameter, e.g., tumor growth, can be evaluated in an animal model system predictive of efficacy in tumor growth. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount can be, but is not necessarily, less than the therapeutically effective amount.

Also provided herein are kits comprising compositions, cells, vectors, nucleic acid molecules, or polypeptides as described herein. The kit can include one or more other elements including: instructions for use; other reagents, e.g., a label, a therapeutic agent, or an agent useful for chelating, or otherwise coupling, a molecule to a label or other therapeutic agent, or a radioprotective composition; devices or other materials for preparing the molecule for administration; pharmaceutically acceptable carriers; and devices or other materials for administration to a subject.

### Vasopressors

In some embodiments the pharmaceutical composition comprising the polypeptide is administered subsequent to, prior to, or in combination with a vasopressor. In some embodiments, the subject in need thereof is not administered a vasopressor or the subject has been previously administered a lower amount of a vasopressor, is concurrently administered a lower amount of a vasopressor, or will subsequently be administered a lower amount of a vasopressor than the amount of a vasopressor administered in the absence of the pharmaceutical composition. In some embodiments, the subject in need is administered about 5%, 10%, 15%, 20%, 25%, 30%, 35%, 40%, 45%, 50%, 55%, 60%, 65%, 70%, 75%, 80%, 85%, 90%, 95%, or 100%, or between about 5-10%, 10-20%, 20-30%, 30-40%, 40-50%, 50-60%, 60-70%, 70-80%, 80-90%, or 90-100% less vasopressor than a subject not administered the pharmaceutical composition comprising the polypeptide. In some embodiments, the subject in need is administered 1-fold less, 2-fold less, 3-fold less, 4-fold less, 5-fold less, 6-fold less, 7-fold less, 8-fold less, 9-fold less, or 10-fold less vasopressor than a subject not administered the pharmaceutical composition comprising the polypeptide.

Exemplary vasopressors include, but are not limited to, Vasopressin (Pitressin® or Vasostrict®), Phenylephrine (Biorphen® or Vazculep®), epinephrine, norepinephrine, droxidopa, phenylephrine, ephedrine, dobutamine, Dopamine, Angiotensin-II, and Terlipressin.

### Methods

“Treatment” of any disease mentioned herein encompasses an alleviation of at least one symptom of the disease, a reduction in the severity of the disease, or the delay or prevention of disease progression to more serious symptoms that may, in some cases, accompany the disease or to at least one other disease. Treatment need not mean that the disease is totally cured. A useful therapeutic agent needs only to reduce the severity of a disease, reduce the severity of symptom(s) associated with the disease or its treatment, or delay the onset of more serious symptoms or a more serious disease that can occur with some frequency following the treated condition. For example, if the disease is a tumor, the composition may reduce the growth or spread of the tumor, or the tumors effect on the tissue in which it is present. A patient's condition can be assessed by standard techniques. Suitable procedures vary according to the patient's condition and symptoms.

In some embodiments, the compositions provided for herein can be used to treat septic shock. In some embodiments, the methods comprise administering to the patient a polypeptide as provided for herein. In some embodiments, the methods comprise administering to the patient an effective amount of a pharmaceutical composition comprising a polypeptide as provided for herein.

In some embodiments, a method of treating septic shock in a subject in need thereof is provided, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
   wherein:
X¹ is E or is absent;
X² is L or is absent;
X³ is Q or is absent;
X⁴ is A, T, or is absent;
X⁵ is T, E, or is absent;
X⁶ is H or Y;
X⁷ is D or E;
X⁸ is F or I; and
X⁹ is R or K; or
   a polypeptide comprising an amino acid sequence selected from the group consisting of SEQ ID NOs: 10, 11, 12, 13, 14, 15, 17, 18, 19, 20, 21, 22, 24, 25, 26, 27, 28, 29, or 30; and a pharmaceutically acceptable carrier.

In some embodiments, the polypeptide is at least 85%, 90%, 91%, 92%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30. In some embodiments, the polypeptide comprises SEQ ID NOs: 2, 3, or 4. In some embodiments, the polypeptide consists of SEQ ID NOs: 2, 3, or 4.

In some embodiments, the subject in need thereof has been diagnosed with sepsis and has a low blood pressure that is not alleviated by the administration of intravenous fluids alone. In some embodiments, the subject has a mean arterial pressure (MAP) of less than 65 mm Hg. In some embodiments, the subject has a mean arterial pressure (MAP) of less than 60 mm Hg. In some embodiments, the subject has a mean arterial pressure (MAP) of less than 55 mm Hg. In some embodiments, the subject has a decrease in mean arterial pressure or systolic blood pressure of about 44 mm Hg or more. In some embodiments, the subject has blood lactate levels that are greater than about 2 mmol. In some embodiments, the subject has blood lactate levels that are greater than about 4 mmol. In some embodiments, the subject has blood lactate levels that are greater than about 6 mmol. In some embodiments, the subject has blood lactate levels that are greater than about 8 mmol. In some embodiments, the subject has blood lactate levels that are greater than about 10 mmol. In some embodiments, the subject has persistent signs of multiple organ damage or failure. In some embodiments, the subject has been diagnosed with septic shock.

In some embodiments, also provided is a method of treating a subject with a significant drop in MAP, the method comprising administer to the subject an effective amount of any pharmaceutical composition described herein. In some embodiments, the subject has a decrease in MAP or systolic blood pressure of about 44 mm Hg or more and has been diagnosed with sepsis. In some embodiments, the subject has been diagnosed with sepsis and has a low blood pressure that is not alleviated by the administration of intravenous fluids alone. In some embodiments, the subject has a MAP of less than about 65 mm Hg. In some embodiments, the subject has blood lactate levels that are greater than about 4 mmol. In some embodiments, the subject has persistent signs of multiple organ damage or failure. In some embodiments, the subject has been diagnosed with septic shock.

In some embodiments, the subject maintains a normal blood oxygen level after administration of the pharmaceutical composition. A normal blood oxygen level is generally 95%-100%. In some embodiments, the subject maintains a blood oxygen level of 95-100% after administration of the pharmaceutical composition. In some embodiments, the subject's blood oxygen level does not decrease by more than 95%, 90%, 85%, 80%, 75%, 70%, 65%, 60%, 55%, 50%, or more after administration of the pharmaceutical composition. In some embodiments, the subject's blood oxygen level increases after administration of the pharmaceutical composition. In some embodiments, the subject's blood oxygen level increases by 1%, 5%, 10%, 15%, 20%, 25%, 30%, 35%, 40%, 45%, 50%, or more, after administration of the pharmaceutical composition.

In some embodiments, the pharmaceutically acceptable carrier is any pharmaceutically acceptable carrier disclosed herein. In some embodiments, the pharmaceutically acceptable carrier is water.

To treat the disease of interest, the compositions and polypeptides described herein can be administered by any appropriate method including, but not limited to, parenteral, topical, oral, nasal, vaginal, rectal, or pulmonary (by inhalation) administration. If injected, the composition(s) can be administered intra-articularly, intravenously, intraarterially, intramuscularly, intravesicularly, intraperitoneally, intratracheally or subcutaneously by bolus injection or continuous infusion. Localized administration, that is, at the site of disease, is contemplated, as are transdermal delivery and sustained release from implants, skin patches, or suppositories. Delivery by inhalation includes, for example, nasal or oral inhalation, use of a nebulizer, inhalation in aerosol form, and the like. Administration via a suppository inserted into a body cavity can be accomplished, for example, by inserting a solid form of the composition in a chosen body cavity and allowing it to dissolve. Other alternatives include eyedrops, oral preparations such as pills, lozenges, syrups, and chewing gum, and topical preparations such as lotions, gels, sprays, and ointments.

In the performance of the methods of treatment, the compositions described herein can be administered as described herein and above. For example, the composition can be administered at any dosage, frequency, and duration that can be effective to treat the condition being treated. The dosage depends on the molecular nature of the active ingredient and the nature of the disorder being treated. Treatment may be continued as long as necessary to achieve the desired results. The compositions provided for herein can be administered as a single dosage or as a series of dosages given periodically, including multiple times per day, daily, every other day, twice a week, three times per week, weekly, every other week, and monthly dosages, among other possible dosage regimens. The periodicity of treatment may or may not be constant throughout the duration of the treatment. For example, treatment may initially occur at weekly intervals and later occur every other week. Treatments having durations of days, weeks, months, or years are encompassed by the embodiments provided for herein. Treatment may be discontinued and then restarted. Maintenance doses may or may not be administered after an initial treatment.

Dosage may be measured as milligrams per kilogram of body weight (mg/kg) or as milligrams per square meter of skin surface (mg/m²) or as a fixed dose, irrespective of height or weight. All of these are standard dosage units in the art. A person's skin surface area is calculated from her height and weight using a standard formula.

As used herein, the phrase “in need thereof” means that the subject (animal or mammal) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

### Embodiments

In some embodiments, embodiments provided herein also include, but are not limited to:

1. A method of treating septic shock in a subject in need thereof, the method comprising administering to the subject an effective amount of a pharmaceutical composition comprising a polypeptide comprising an amino acid sequence of:
X1X2X3X4X5LX6X7X8X9HQIL (SEQ ID NO: 1)
wherein:
   X1 is E or is absent;
   X2 is L or is absent;
   X3 is Q or is absent;
   X4 is A, T, or is absent;
   X5 is T, E, or is absent;
   X6 is H or Y;
   X7 is D or E;
   X8 is F or I; and
   X9 is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier.

2. The method of embodiment 1, wherein the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

3. The method of embodiment 1, wherein the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

4. The method of embodiment 1, wherein the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

5. The method of embodiment 1, wherein the polypeptide comprises SEQ ID NO: 2.

6. The method of embodiment 1, wherein the polypeptide consists of SEQ ID NO: 2.

7. The method of embodiment 1, wherein the polypeptide comprises SEQ ID NO: 3.

8. The method of embodiment 1, wherein the polypeptide consists of SEQ ID NO: 3.

9. The method of embodiment 1, wherein the polypeptide comprises SEQ ID NO: 4.

10. The method of embodiment 1, wherein the polypeptide consists of SEQ ID NO: 4.

11. The method of any one of the preceding embodiments, wherein the pharmaceutical composition comprises a liposome encapsulated polypeptide.

12. The method of any one of the preceding embodiments, wherein the pharmaceutically acceptable carrier is any pharmaceutically acceptable carrier disclosed herein.

13. The method of any one of the preceding embodiments, wherein the pharmaceutical composition is administered to the subject by any administration method disclosed herein.

14. The method of any one of the preceding embodiments, wherein the pharmaceutical composition is administered to the subject by aerosol inhalation, intratracheal injection, or intravenous injection.

15. The method of any one of the preceding embodiments, wherein the subject in need thereof has been diagnosed with sepsis and has low blood pressure that is not alleviated by the administration of intravenous fluids alone.

16. The method of embodiment 15, wherein the subject in need thereof has a mean arterial pressure (MAP) less than about 65 mm Hg.

17. The method of any one of the preceding embodiments, wherein the subject in need thereof has been diagnosed with sepsis and has a decrease in mean arterial pressure or systolic blood pressure of about 44 mm Hg or more.

18. The method of any one of embodiments 15-17, wherein the subject in need thereof has blood lactate levels that are greater than about 4 mmol.

19. The method of any one of embodiments 15-17, wherein the subject in need thereof has persistent signs of multiple organ damage or failure.

20. The method of any one of the preceding embodiments, wherein the subject's blood oxygen level does not decrease after administration of the pharmaceutical composition.

21. The method of embodiment 20, wherein the subject's blood oxygen level does not decrease more than 50% after administration of the pharmaceutical composition.

22. The method of embodiment 20, wherein the subject's blood oxygen level does not decrease more than 65% after administration of the pharmaceutical composition.

23. The method of any one of the preceding embodiments, wherein the subject's blood oxygen level increases after administration of the pharmaceutical composition.

24. The method of any one of the preceding embodiments, wherein the subject maintains a normal blood oxygen level after administration of the pharmaceutical composition.

25. The method of any one of the preceding embodiments, wherein the subject in need thereof is not administered a vasopressor or the subject has been previously administered a lower amount of a vasopressor, is concurrently administered a lower amount of a vasopressor, or will subsequently be administered a lower amount of a vasopressor than the amount of a vasopressor administered in the absence of the pharmaceutical composition.

26. The method of any one of the preceding embodiments, wherein the subject in need thereof has been diagnosed with septic shock.

27. A method of treating subject with a significant drop in mean arterial pressure, the method comprising administering to the subject an effective amount of a pharmaceutical composition comprising a polypeptide comprising an amino acid sequence of:
X1X2X3X4X5LX6X7X8X9HQIL (SEQ ID NO: 1)
wherein:
   X1 is E or is absent;
   X2 is L or is absent;
   X3 is Q or is absent;
   X4 is A, T, or is absent;
   X5 is T, E, or is absent;
   X6 is H or Y;
   X7 is D or E;
   X8 is F or I; and
   X9 is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier.

28. The method of embodiment 27, wherein the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

29. The method of embodiment 27, wherein the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

30. The method of embodiment 27, wherein the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

31. The method of embodiment 27, wherein the polypeptide comprises SEQ ID NO: 2.

32. The method of embodiment 27, wherein the polypeptide consists of SEQ ID NO: 2.

33. The method of embodiment 27, wherein the polypeptide comprises SEQ ID NO: 3.

34. The method of embodiment 27, wherein the polypeptide consists of SEQ ID NO: 3.

35. The method of embodiment 27, wherein the polypeptide comprises SEQ ID NO: 4.

36. The method of embodiment 27, wherein the polypeptide consists of SEQ ID NO: 4.

37. The method of any one of the preceding embodiments, wherein the pharmaceutical composition comprises a liposome encapsulated polypeptide.

38. The method of any one of the preceding embodiments, wherein the pharmaceutically acceptable carrier is any pharmaceutically acceptable carrier disclosed herein.

39. The method of any one of the preceding embodiments, wherein the pharmaceutical composition is administered to the subject by any administration method disclosed herein.

40. The method of any one of the preceding embodiments, wherein the pharmaceutical composition is administered to the subject by aerosol inhalation, intratracheal injection, or intravenous injection.

41. The method of any one of the preceding embodiments, wherein the subject has a decrease in mean arterial pressure or systolic blood pressure of about 44 mm Hg or more and has been diagnosed with sepsis.

42. The method of any one of the preceding embodiments, wherein the subject has been diagnosed with sepsis and has low blood pressure that is not alleviated by the administration of intravenous fluids alone.

43. The method of embodiment 42, wherein the subject has a mean arterial pressure (MAP) less than about 65 mm Hg.

44. The method of any one of embodiments 41-43, wherein the subject in need thereof has blood lactate levels that are greater than about 4 mmol.

45. The method of any one of embodiments 41-43, wherein the subject in need thereof has persistent signs of multiple organ damage or failure.

46. The method of any one of the preceding embodiments, wherein the subject's blood oxygen level does not decrease after administration of the pharmaceutical composition.

47. The method of embodiment 46, wherein the subject's blood oxygen level does not decrease more than 50% after administration of the pharmaceutical composition.

48. The method of embodiment 46, wherein the subject's blood oxygen level does not decrease more than 65% after administration of the pharmaceutical composition.

49. The method of any one of the preceding embodiments, wherein the subject's blood oxygen level increases after administration of the pharmaceutical composition.

50. The method of any one of the preceding embodiments, wherein the subject maintains a normal blood oxygen level after administration of the pharmaceutical composition.

51. The method of any one of the preceding embodiments, wherein the subject in need thereof is not administered a vasopressor or the subject has been previously administered, is concurrently administered, or will subsequently be administered a lower amount of a vasopressor than the amount of a vasopressor administered in the absence of the pharmaceutical composition.

52. The method of any one of the preceding embodiments, wherein the subject in need thereof has been diagnosed with septic shock.

53. A method of preventing heart damage in a subject with diagnosed with septic shock, the method comprising administering to the subject an effective amount of a pharmaceutical composition comprising a polypeptide comprising an amino acid sequence of:
X1X2X3X4X5LX6X7X8X9HQIL (SEQ ID NO: 1)
wherein:
   X1 is E or is absent;
   X2 is L or is absent;
   X3 is Q or is absent;
   X4 is A, T, or is absent;
   X5 is T, E, or is absent;
   X6 is H or Y;
   X7 is D or E;
   X8 is F or I; and
   X9 is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier, as compared to a subject not treated with the pharmaceutical composition.

54. The method of embodiment 53, wherein the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

55. The method of embodiment 53, wherein the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

56. The method of embodiment 53, wherein the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

57. The method of embodiment 53, wherein the polypeptide comprises SEQ ID NO: 2.

58. The method of embodiment 53, wherein the polypeptide consists of SEQ ID NO: 2.

59. The method of embodiment 53, wherein the polypeptide comprises SEQ ID NO: 3.

60. The method of embodiment 53, wherein the polypeptide consists of SEQ ID NO: 3.

61. The method of embodiment 53, wherein the polypeptide comprises SEQ ID NO: 4.

62. The method of embodiment 53, wherein the polypeptide consists of SEQ ID NO: 4.

63. The method of any one of the preceding embodiments, wherein the pharmaceutical composition comprises a liposome encapsulated polypeptide.

64. The method of any one of the preceding embodiments, wherein the pharmaceutically acceptable carrier is any pharmaceutically acceptable carrier disclosed herein.

65. The method of any one of the preceding embodiments, wherein the pharmaceutical composition is administered to the subject by any administration method disclosed herein.

66. The method of any one of the preceding embodiments, wherein the pharmaceutical composition is administered to the subject by aerosol inhalation, intratracheal injection, or intravenous injection.

67. The method of any one of the preceding embodiments, wherein the subject in need thereof has low blood pressure that is not alleviated by the administration of intravenous fluids alone.

68. The method of embodiment 67, wherein the subject in need thereof has a mean arterial pressure (MAP) less than about 65 mm Hg.

69. The method of any one of the preceding embodiments, wherein the subject in need thereof has a decrease in mean arterial pressure or systolic blood pressure of about 44 mm Hg or more.

70. The method of any one of embodiments 67-69, wherein the subject in need thereof has blood lactate levels that are greater than about 4 mmol.

71. The method of any one of embodiments 67-69, wherein the subject in need thereof has persistent signs of multiple organ damage or failure.

72. The method of any one of the preceding embodiments, wherein the subject's blood oxygen level does not decrease after administration of the pharmaceutical composition.

73. The method of embodiment 72, wherein the subject's blood oxygen level does not decrease more than 50% after administration of the pharmaceutical composition.

74. The method of embodiment 72, wherein the subject's blood oxygen level does not decrease more than 65% after administration of the pharmaceutical composition.

75. The method of any one of the preceding embodiments, wherein the subject's blood oxygen level increases after administration of the pharmaceutical composition.

76. The method of any one of the preceding embodiments, wherein the subject maintains a normal blood oxygen level after administration of the pharmaceutical composition.

77. The method of any one of the preceding embodiments, wherein the subject in need thereof has a troponin level that that does not increase after administration of the pharmaceutical composition.

78. The method of any one of the preceding embodiments, wherein the subject in need thereof maintains normal troponin levels after administration of the pharmaceutical composition.

79. The method of any one of the preceding embodiments, wherein the subject in need thereof is not administered a vasopressor or the subject has been previously administered, is concurrently administered, or will subsequently be administered a lower amount of a vasopressor than the amount of a vasopressor administered in the absence of the pharmaceutical composition.

80. A method of increasing survival of a subject with diagnosed with septic shock, the method comprising administering to the subject an effective amount of a pharmaceutical composition comprising a polypeptide comprising an amino acid sequence of:
X1X2X3X4X5LX6X7X8X9HQIL (SEQ ID NO: 1)
wherein:
   X1 is E or is absent;
   X2 is L or is absent;
   X3 is Q or is absent;
   X4 is A, T, or is absent;
   X5 is T, E, or is absent;
   X6 is H or Y;
   X7 is D or E;
   X8 is F or I; and
   X9 is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier, as compared to a subject not treated with the pharmaceutical composition.

81. The method of embodiment 80, wherein the polypeptide comprises an amino acid sequence that is at least 85% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

82. The method of embodiment 80, wherein the polypeptide comprises an amino acid sequence that is at least 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

83. The method of embodiment 80, wherein the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

84. The method of embodiment 80, wherein the polypeptide comprises SEQ ID NO: 2.

85. The method of embodiment 80, wherein the polypeptide consists of SEQ ID NO: 2.

86. The method of embodiment 80, wherein the polypeptide comprises SEQ ID NO: 3.

87. The method of embodiment 80, wherein the polypeptide consists of SEQ ID NO: 3.

88. The method of embodiment 80, wherein the polypeptide comprises SEQ ID NO: 4.

89. The method of embodiment 80, wherein the polypeptide consists of SEQ ID NO: 4.

90. The method of any one of the preceding embodiments, wherein the pharmaceutical composition comprises a liposome encapsulated polypeptide.

91. The method of any one of the preceding embodiments, wherein the pharmaceutically acceptable carrier is any pharmaceutically acceptable carrier disclosed herein.

92. The method of any one of the preceding embodiments, wherein the pharmaceutical composition is administered to the subject by any administration method disclosed herein.

93. The method of any one of the preceding embodiments, wherein the pharmaceutical composition is administered to the subject by aerosol inhalation, intratracheal injection, or intravenous injection.

94. The method of any one of the preceding embodiments, wherein the subject in need thereof has been diagnosed with sepsis and has low blood pressure that is not alleviated by the administration of intravenous fluids alone.

95. The method of embodiment 94, wherein the subject in need thereof has a mean arterial pressure (MAP) less than about 65 mm Hg.

96. The method of any one of the preceding embodiments, wherein the subject in need thereof has a decrease in mean arterial pressure or systolic blood pressure of about 44 mm Hg or more.

97. The method of any one of embodiments 94-96, wherein the subject in need thereof has blood lactate levels that are greater than about 4 mmol.

98. The method of any one of embodiments 94-96, wherein the subject in need thereof has persistent signs of multiple organ damage or failure.

99. The method of any one of the preceding embodiments, wherein the subject's blood oxygen level does not decrease after administration of the pharmaceutical composition.

100. The method of embodiment 99, wherein the subject's blood oxygen level does not decrease more than 50% after administration of the pharmaceutical composition.

101. The method of embodiment 99, wherein the subject's blood oxygen level does not decrease more than 65% after administration of the pharmaceutical composition.

102. The method of any one of the preceding embodiments, wherein the subject's blood oxygen level increases after administration of the pharmaceutical composition.

103. The method of any one of the preceding embodiments, wherein the subject maintains a normal blood oxygen level after administration of the pharmaceutical composition.

104. The method of any one of the preceding embodiments, wherein the subject in need thereof is not administered a vasopressor or the subject has been previously administered, is concurrently administered, or will subsequently be administered a lower amount of a vasopressor than the amount of a vasopressor administered in the absence of the pharmaceutical composition.

### EXAMPLES

The following examples are illustrative, but not limiting, of the compounds, compositions and methods described herein. Other suitable modifications and adaptations known to those skilled in the art are within the scope of the embodiments provided for herein.

### Example 1: Inhibition of aiPLA2 Activity of Human Prdx6 by Select Polypeptides

The polypeptides disclosed herein are based in part on the engineering of specific peptide inhibitors of aiPLA2. Tables 2, 3, and 4 below show the effect of select polypeptides on the aiPLA2 activity of recombinant human Prdx6 (Table 2), the size optimization of inhibitory polypeptides by the effect on aiPLA2 activity of human recombinant protein (Table 3), and the effect of select substitutions in PIP-2 inhibition of aiPLA2 activity of human recombinant Prdx6 (Table 4).

TABLE 2
Effect of peptides on aiPLA2
activity of recombinant human Prdx6

| | aiPLA2 |
|---|---|
| | activity |
| | (nmol/ |
| Sequence | min/mg) |
| DEELQATLHDFRHQIL | 45.0 |
| (human PIP-1, SEQ ID NO: 5) | |
| DEELQTELYEIKHQIL | 32.0 |
| (rat, mouse PIP-3, SEQ ID NO: 6) | |
| ELQTELYEIKHQIL (SEQ ID NO: 7) | 33.2 |
| QTELYEIKHQIL (SEQ ID NO: 8) | 31.5 |
| ELYEIKHQIL (SEQ ID NO: 9) | 28.6 |
| YEIKHQIL (SEQ ID NO: 10) | 94.4 |
| IKHQIL (SEQ ID NO: 11) | 97.1 |
| DEELQTELYEIKHQ (SEQ ID NO: 12) | 102 |
| DEELQTELYEIK (SEQ ID NO: 13) | 95.7 |
| DEELQTELYEI (SEQ ID NO: 14) | 94.9 |
| DEELQTEL (SEQ ID NO: 15) | 93.6 |
| human Prdx6 only (no polypeptide) | 93.4 |
| DEELQATLHDFRHQIL | 20.1 |
| (human PIP-1, SEQ ID NO: 5) | |

TABLE 3
Size optimization of inhibitory
polypeptide by effect on aiPLA2 activity
of human recombinant protein

| | aiPLA2 |
|---|---|
| | activity |
| | (nmol/ |
| Sequence | min/mg) |
| TLHDFRHQIL (SEQ ID NO: 16) | 31.5 |
| LHDFRHQIL (SEQ ID NO: 2) | 29.9 |
| TLHDFRHQI (SEQ ID NO: 17) | 89.6 |
| LHDFRHQI (SEQ ID NO: 18) | 93.6 |
| LYEIKHQIL (SEQ ID NO: 3) | 32.3 |
| ELYEIKHQI (SEQ ID NO: 19) | 93.5 |
| LYEIKHQI (SEQ ID NO: 20) | 92.1 |
| human Prdx6 only (no polypeptide) | 92.0 |
| DEELQATLHDFRHQIL | 32.0 |
| (human PIP-1, SEQ ID NO: 5) | |

TABLE 4
Substitutions in PIP-2:
effect on inhibition of aiPLA2
activity of recombinant human Prdx6

| | aiPLA2 |
|---|---|
| | activity |
| | (nmol/ |
| Sequence | min/mg) |
| LKIEYHQIL (SEQ ID NO: 21) | 97.67 |
| LRFDHHQIL (SEQ ID NO: 22) | 98.97 |
| LYDIRHQIL (SEQ ID NO: 4) | 29.74 |
| LHEFKHQIL (SEQ ID NO: 23) | 99.42 |
| LFKLEHQIL (SEQ ID NO: 24) | 97.95 |
| LHDFRDQIL (SEQ ID NO: 25) | 102.04 |
| LHDFRPQIL (SEQ ID NO: 26) | 101.34 |
| LHDFRHNIL (SEQ ID NO: 27) | 98.97 |
| LHDFRHIIL (SEQ ID NO: 28) | 97.30 |
| LHDFRHQLL (SEQ ID NO: 29) | 102.45 |
| LHDFRHQTL (SEQ ID NO: 30) | 100.35 |
| human Prdx6 only (no polypeptide) | 99.91 |
| LHDFRHQIL (SEQ ID NO: 2) | 26.18 |

### Example 2: Treatment of Septic Shock Induced Organ Injury

In order to determine the effects of PIP-2 in the treatment of acute lung injury and other organ damage resulting from septic shock, healthy adolescent Yorkshire porcine subjects were separated in the three groups: (Group 1) healthy control plus standard of care treatment with mechanical ventilation (negative control); (Group 2) placebo treatment plus standard of care treatment with mechanical ventilation, fluids, glucose, and vasopressors (positive control); and (Group 3) PIP-2 treatment plus standard of care treatment the same as group (2). Both arms of the subjects were dosed in a bolus intravenous push, with the bolus for groups (2) and (3) containing bacterial lipopolysaccharide (LPS) to induce septic shock. Total study duration was 8 hours from injection.

LPS induced severe and rapid septic shock in the subjects, with the worse-case scenario being death in 4-7 hours in LPS administration. Additionally, the septic shock resulted in protein build-up in the lung lavage as a result of a damaged alveolar-capillary barrier; myeloperoxidase (MPO) in the lung lavage which reflected increased white cell counts; a “cytokine storm” that was a result of immune over-activation, leading to injury; and increased troponin levels as a result of heart damage.

Results showed that treatment with PIP-2 lowered the protein located in the lung lavage to that of the negative control group, as shown in Table 5.

TABLE 5
Protein In Lung Lavage

| Group | Mean Protein mg/Kg | Number |
|---|---|---|
| Healthy Control | 1.5 | n = 4 |
| Placebo (LPS control) | 5.0 | n = 6 |
| PIP-2 | 1.4 | n = 9 |

Results also showed that treatment with PIP-2 protects from inflammation and white cell activation by lowering MPO levels, which is an indicator of lung inflammation and immune response to injury, as shown in Table 6.

TABLE 6
MPO levels

| Group | Mean Protein ng/mg | Number |
|---|---|---|
| Healthy Control | 5 | n = 5 |
| Placebo (LPS control) | 46 | n = 6 |
| PIP-2 | 8 | n = 9 |

Further, treatment with PIP-2 also prevented the “cytokine storm” in a measurement of three different cytokine levels in the bronchoalveolar lavage fluid (BALf), as shown in Table 7. High cytokine levels in the BALf indicated activation of lung immune responses to injury.

TABLE 7
Cytokine Analysis of BALf

| | Mean Protein pg/mg | | | |
|---|---|---|---|---|
| Group | IL-6 | IL-1β | TNF-α | Number |
| Healthy Control | 263 | 107 | 94 | Healthy Control |
| Placebo (LPS control) | 550 | 238 | 199 | Placebo (LPS control) |
| PIP-2 | 283 | 133 | 103 | PIP-2 |

Animals treated with PIP-2 also preformed drastically better on the Horowitz Index, which is a measurement used to assess lung function and damage in patients, especially those on ventilators. On the Horowitz Index, a score greater than 300 is normal, a score between 201-300 indicates mild lung damage, a score between 100-200 indicates moderate lung damage, and a score less than 100 indicates severe lung damage. The Horowitz Index scores for the placebo (LPS control) and PIP-2 treated groups is shown in Table 8. The PIP-2 treated subjects were more than twice as likely to have normal lung functions, and half as likely to die.

TABLE 8
Horowitz Index Scores

| Group | Index Score |
|---|---|
| Placebo (LPS Control) | |
| Normal | 17% |
| Moderate | 33% |
| Death | 50% |
| PIP-2 Treated | |
| Normal | 50% |
| Mild | 8% |
| Moderate | 8% |
| Severe | 8% |
| Death | 25% |

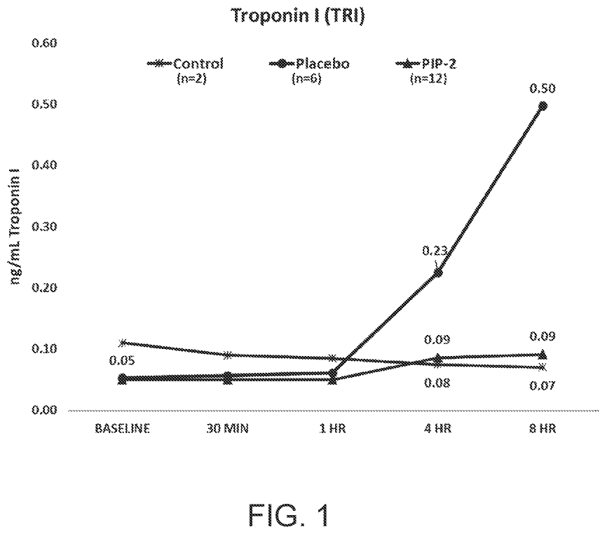

In addition to the positive results in the lung, treatment with PIP-2 also provided positive results in the heart and vasculature of the subjects. First, troponin in the blood is an indication of heart damage, because the protein is normally not present in the blood but leaks into the bloodstream when heart muscles are damaged, which can occur in septic shock. As shown in FIG. 1, troponin levels in the placebo LPS control group began to sharply rise from 1 to 8 hours in the study, with a significant difference observed at 4 hours. However, the PIP-2 treated group showed no such rise and remained level with the control group.

Second, treatment with PIP-2 also provided protection against creatine kinase (CK) protein levels in the blood. Like troponin, CK protein leaks in to the bloodstream when heart muscles and other skeletal muscles are damaged. In a one-timepoint measurement, control subjects had a mean CK level of 19% (U/mL), while placebo treated LPS controls had a much higher 89% CK level. Subjects treated with PIP-2 however had the CK level drop to about 15%. Therefore, PIP-2 protected the animals from the LPS induced septic shock.

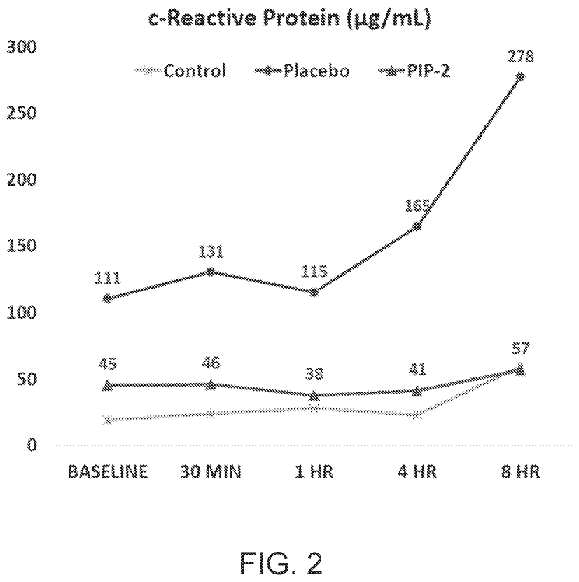

Similarly, treatment with PIP-2 also prevented the increase of c-reactive protein (CRP) an indicator of inflammation. As shown in FIG. 2, CRP levels in the placebo LPS control group began to sharply rise from 1 to 8 hours, similar to what occurred with troponin levels. The PIP-2 treated group again showed no such rise and CRP levels remained low. A statistically significant difference in the c-reactive protein blood levels between the PIP-2 treated group and the placebo group was observed at 8 hours.

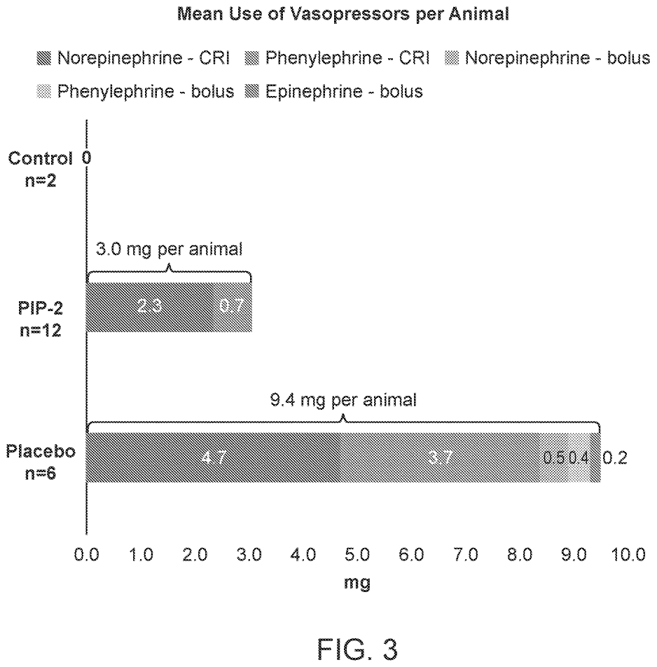

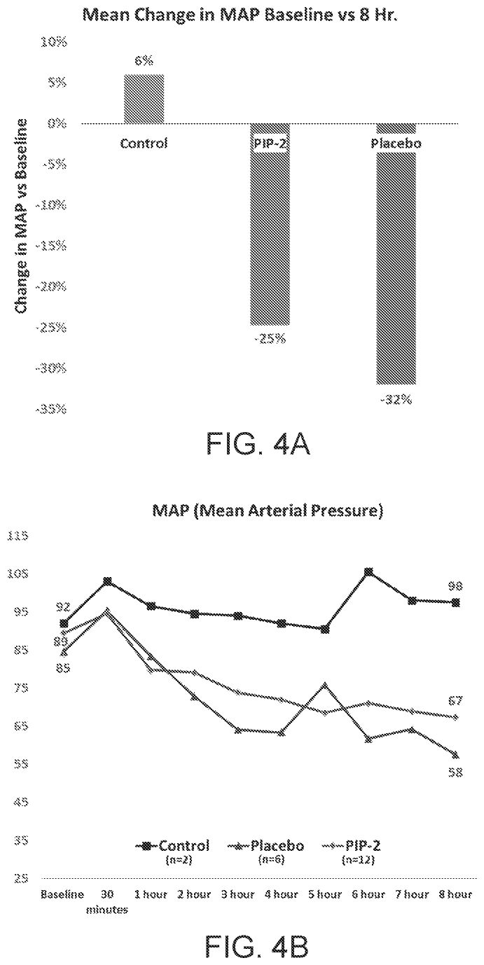

Septic shock caused a critical drop in mean arterial pressure (MAP), which is one of the major causes of death for the condition. Since septic shock patients do not respond with an increase in MAP with just IV fluids alone, vasopressor treatment is a primary tool to increase MAP and stabilize blood flow. As shown in FIG. 3, placebo LPS control group subjects needed treatment with rescue vasopressors as well as continuous infusion in an attempt to stabilize MAP. In comparison, PIP-2 treated subjects required about 66% less vasopressor treatment and little to no rescue treatment. Additionally, PIP-2 treated subjects had better overall MAP even without the use of vasopressors. Measuring the mean change in MAP from the baseline start of the study to the 8 hour conclusion, the placebo LPS control group had an average of a −32% MAP decrease, while the PIP-2 treated subjects had only a −25% MAP decrease (FIGS. 4A and 4B).

In summary, in this 8 hour LPS induced septic shock model, treatment with PIP-2 showed superior outcomes across several critical measurements. PIP-2 treatment reduced septic shock mortality by half, while 50% of the treated subjects showed complete protection. Further PIP-2 treatment unexpectedly showed several benefits, including lower protein levels in the lung, lower troponin, creatine kinase, and CRP levels in the blood, reduced use of vasopressors to stability MAP, and overall improved cardiovascular performance.

### Example 3: Decreased Lipopolysaccharide (LPS)-Induced Acute Lung Injury in Pigs by Treatment with a Nonapeptide (PIP-2) that Inhibited the Phospholipase A2 Activity of Peroxiredoxin 6

The efficacy of a liposome-encapsulated 9 amino acid peptide (PIP-2) for the prevention/treatment of lipopolysaccharide (LPS)-induced sepsis and acute lung injury (ALI) was addressed in pigs. PIP-2 inhibits the PLA2 activity of peroxiredoxin 6, thereby preventing the activation of NADPH oxidases, types 1 and 2. Twenty Yorkshire female pigs were infused intravenously with liposomes alone (control, n=2), LPS+liposomes (untreated, n=6), and LPS+PIP-2 in liposomes (treated, n=12). Animals were mechanically ventilated and were euthanized after 8 hours or earlier if pre-established humane endpoints were reached. Control pigs were essentially unchanged over the 8-hour study. LPS administration resulted in systemic inflammation with manifestations of clinical sepsis, decreased lung function with a marked decrease in the arterial PO2, and vascular instability resulting in early euthanasia of 50% of untreated animals. PIP-2 treatment reduced the requirement for supportive vasopressors and the manifestations of lung injury so that only 25% of animals required early euthanasia. Broncho-alveolar lavage fluid showed markedly lower total protein, cytokines (TNF-α, IL-6, and IL-1β), and myeloperoxidase levels in PIP-2-treated as compared to untreated pigs. Thus, the porcine LPS-induced sepsis model was associated with moderate to severe lung pathophysiology compatible with ALI. Treatment with PIP-2 markedly decreased lung injury, cardiovascular instability, and early sacrifice of pigs. These results indicated that inhibition of ROS production via NOX1/2 had a beneficial effect in treating pigs with LPS-induced sepsis and ALI. Accordingly, PIP-2 and the peptides provided for herein can be used to treat septic shock and injuries related to the same, such as ALI.

Acute respiratory distress syndrome (ARDS) and acute lung injury (ALI) can be associated with a variety of pulmonary (e.g., aspiration, pneumonia) or non-pulmonary (e.g., sepsis, trauma) etiologies. The physiological effects associated with ARDS include stiff lungs (decreased lung compliance), lung edema, and progressive hypoxemia. The current standard of care is based on treatment of the underlying pathology and respiratory support using controlled ventilation that avoids mechanical injury to lung tissue. But, despite optimal respiratory support, ARDS currently has a mortality rate of ~40%.

Although the causes of ALI are multifactorial, it is now recognized that the excessive release of reactive oxygen species (ROS) including oxygen-derived radicals can play an important role in lung injury associated with many different etiologies. NADPH oxidases (NOX) are a major enzymatic source of ROS in the lung. ROS generated by NOX enzymes are crucial for the regulation of important cellular functions such as host defense, cellular signaling, cell migration, cell differentiation, and post-translational protein processing. However, excessive production of ROS can lead to oxidation of tissue macromolecules (lipids, proteins, DNA) resulting in widespread cellular injury. ROS can be produced in the lung by inflammatory cells including polymorphonuclear leukocytes (PMN) and alveolar macrophages (AM) as well as by parenchymal lung cells including both epithelial and endothelial cells. Thus, release of ROS can play an important pathophysiologic role in the manifestations of ALI/ARDS.

The NOX family consists of 7 different proteins. NOX2 is the major NOX enzyme in phagocytic cells and in the lung while NOXes 1 and 2 are predominant in the cardiovascular system. NOX2 is a complex of 2 cell membrane-associated protein components, gp91^{phox} and p22^{phox}, that is inactive in the resting state. Activation of the enzyme in cells involves phosphorylation of gp91^{phox}, the translocation to the membrane of 3 additional cytosolic proteins (p67^{phox}, p47^{phox}, p40^{phox}), and the activation of cytosolic rac protein (either rac1 or rac2). Rac is a member of the Rho family of GTPases; rac1 is the activating co-factor in parenchymal lung cells while rac2 is required in PMN. The protein peroxiredoxin 6 (Prdx6) has an important role in ROS production via NOX2 since it is required for activation of rac. Thus, the activation process requires the phospholipase A2 activity (aiPLA2) of Prdx6 that modulates rac release through lysophosphatidic acid receptor signaling. NOX1 also requires rac protein for activation of ROS production but the other 5 NOX enzymes are independent of rac.

The lipid compound termed MJ33 and the surfactant protein A (SP-A) are 2 agents that bind to Prdx6 preventing aiPLA2 activity, in turn resulting in failure of rac activation. A 9 amino acid sequence of SP-A was identified as being responsible for inhibition of NOX2 activation. This 9 amino acid peptide sequence is generally conserved in mammals with some minor variation is termed peroxiredoxin 6 PLA2 inhibitory peptide (PIP); the peptide corresponding to the human amino acid peptide sequence in SP-A is termed PIP-2.

Many models of experimental ALI have been described including commonly used models associated with the administration of bacterial lipopolysaccharide (LPS). LPS can be administered either intra-tracheally resulting primarily in lung inflammation or by intra-peritoneal (IP) or intravenous (IV) injection resulting in systemic sepsis along with pulmonary inflammation. In a variety of mouse models of ALI, inhibition of NOX2 activation by treatment with MJ33 or PIP-2 or genetic inactivation of aiPLA2 activity can significantly ameliorate lung injury. The study described herein was designed to evaluate the possible protective role for PIP-2 in an animal model with lungs more closely resembling the anatomy and physiology of human lungs.

### Methods

### Agents

The 9 amino acid peptide called PIP-2 was synthesized with a C-terminal HCl group by APeptide, Shanghai, China. The peptide was encapsulated in liposomes for IV delivery. Liposomes were composed of dipalmitoyl phosphatidylcholine (DPPC), egg PC, phosphatidylglycerol (PG), and cholesterol in the molar ratio 0.5, 0.25, 0.10, 0.15; this composition reflects the lipid composition of lung surfactant. Liposomes were stored at −20 degrees C. prior to use. Samples were warmed to room temperature and were used within 3 hours. The PIP-2 encapsulation efficiency for the liposomes was about 15-20%. LPS, Escherichia coli O55:B5, was obtained from Sigma (product Code: L2637); source batch: 12181107 with sub-batches: 0000102731 and 0000119457. LPS was stored at 3-8 deg. C.

### Animals

The model utilized was based on a previously reported study of LPS-induced injury in pigs (Hochhausen N, Orschulik J, Follmann A, Santos S A, Dohmeier H, Leonhardt S, Rossaint R, Czaplik M. Comparison of two experimental ARDS models in pigs using electrical impedance tomography. PLoS One. 2019 Nov. 13; 14(11):e0225218.). Twenty nulliparous female Yorkshire pigs (Sus scrofa domesticus), 2-3 months old, weight 20.2 to 29.4 kg, (average 24.9±2.1 kg) were purchased from Animal Biotech Industries, Danboro, PA. All animals were held in quarantine prior to assignment to the study. Animals were housed under conditions that met or exceeded requirements as set forth in the USDA AWA/AWR and as described in the Guide. Environmental conditions (temperature, relative humidity, and light) within the animal facility were monitored and remained within acceptable conditions. Potable water was provided ad libitum to all animals. Pigs were offered Purina Lab Diet (#5084 Laboratory Porcine Diet Grower) once daily; no chow was offered on the day of the experiments.

The animals were prepared for surgery using accepted standards of veterinary care. Tiletamine-zolazepam (4-6 mg/kg, IM) or buprenorphine (0.01 mg/kg, IM) was administered as a pre-anesthetic. Isoflurane anesthesia (delivered in 100% oxygen) was administered via mask/nose cone until the animal was in a plane of anesthesia that facilitated endotracheal intubation. Once sufficiently anesthetized, the animals were intubated and two IV catheters were placed in peripheral veins for administering supportive IV fluids and propofol and to allow collection of blood samples for monitoring blood gases and electrolytes; a carotid or femoral artery was catheterized to directly monitor arterial blood pressure and to obtain arterial blood samples. An “introducer sheath” was inserted and advanced into each vessel for providing access. Propofol was administered, initially 4-8 mg/kg IV as a bolus followed by ~0.2-0.4 mg/kg/min IV as a continuous infusion; animals were maintained on propofol anesthesia for the remainder of the procedure. An ophthalmic lubricant was applied to the eyes. A Foley catheter was placed in the bladder, under cystoscopic guidance, if necessary, to allow urine collection throughout the experiment. Warm water heating pads were used to help maintain adequate body temperature while under anesthesia; animals with body temperatures below 96° F. were provided warming blankets and warm fluids. Lactic acidosis (serum lactate >10 mM) was treated with sodium bicarbonate, while hypoglycemia was treated with IV dextrose. Hypotension during the experiment was treated with IV administration of norepinephrine and/or phenylephrine.

### Experimental Protocol

Prior to start of the experiment, the battery of systemic, cardiovascular, and pulmonary tests was carried out. These are called baseline measurements. Pigs were infused IV (zero time) with: Group 1, liposomes alone, no LPS (control, n=2); Group 2, LPS+liposomes (ALI untreated, n=6); and Group 3, LPS+PIP-2 in liposomes (ALI treated, n=12). Technical personnel responsible for animal care were “blinded” to the treatment conditions. LPS was given as an IV infusion of 50 ug/kg body weight over the first hour of the experiment. Before settling on the 50 ug/kg dose of LPS, several pigs were treated IV with LPS at 100 ug/kg that resulted in severe cardiovascular instability and early sacrifice. Either liposomes alone (Group 2) or PIP-2 in liposomes (Group 3) was infused as a “slow” bolus at the end of the LPS infusion. Liposomes were given at 2.22 mg/kg body weight. PIP-2 was given at 20 mg/kg body weight. Based on the measured encapsulation efficiency of ~15-20%, the IV administered PIP-2 was ~3-4 mg/kg body weight in liposomes and the remainder was in aqueous solution; presumably, the encapsulated PIP-2 was internalized by cells while the unencapsulated PIP-2 remained extracellular.

Animals were mechanically ventilated for up to 8 hours in a volume-controlled mode using a tidal volume of 8 mL/kg body weight, an inspiratory-expiratory ratio of 1:1, a fraction of inspired oxygen (FiO2) of 1.0, and a positive end-expiratory pressure (PEEP) of 5 cm H₂O. After baseline measurements, assessment of respiratory mechanics, hemodynamics, blood gases, and other blood parameters was performed at hourly intervals. Arterial blood O₂ (aPO₂) was measured with an O₂ electrode. Static lung compliance was measured as the change in pressure at the end of a “breath hold” with a given tidal volume. These hourly measurements were used to assess the state of the animal in order to administer pre-defined supportive therapies, as needed. Blood samples were taken from a peripheral vein and blood hematology analysis was done on site; serum chemistry was done by commercial clinical laboratories (Antech Diagnostics, Levittown, PA, for cardiac markers and Idexx Laboratories, Westbrook, ME, for other assays).

Animals were euthanized prior to the 8 hour duration of study if pre-established humane endpoints that did not respond to basic treatment were met. These endpoints were: arterial pO₂<50 mmHg while breathing 100% O₂ (Horowitz index <50), marked fever (temp. >105 deg.) or hypothermia (temp. <96 deg.), severe hypotension (blood pressure <50 mmHg) while on IV pressor drugs, or severe lactic acidosis (serum lactate persistently >10 mM). Unless noted otherwise, the values for physiological parameters presented in this report are for the final measurement prior to sacrifice.

Animals surviving for 8 hours were subjected to bronchoalveolar lavage (BAL) following the 8 h cardio-respiratory measurements. FITC-dextran was infused IV over 15 minutes and then a bronchoscope was inserted into the airway and navigated into a subsegmental bronchus. Tubing was attached to the working channel of the bronchoscope on one end and to a syringe containing saline through a three-way stopcock on the other end. The third port of the stopcock was attached to a trap to collect the BAL fluid (BALF). The effluent was collected when the stopcock was turned off to the syringe causing suction through the trap. Pig lung lavage (BALf) was centrifuged at 1000×g for 10 min and used for biochemical assays. Protein content was measured using a Bio-Rad assay kit with gamma globulin as standard. Myeloperoxidase (MPO) activity was measured by ELISA assay using a commercial kit (Biomatik, Wilmington DE). Cytokines (IL-6, IL-1β, TNF-α) also were measured by ELISA assay using a commercial assay kit (Invitrogen, ThermoFisher Scientific, Federic MD).

At the conclusion of each experiment, the animal was euthanized via an overdose of euthanasia solution (IV) in accordance with accepted AVMA guidelines. For all animals, the lungs, liver, kidneys, and heart were collected after sacrifice and fixed in 10% formalin, paraffin processed, and stained with hematoxylin and eosin (H&E) for histopathological evaluation.

### Statistical Analysis

Results are presented as mean±standard deviation (SD). Group differences were evaluated by 2-tailed t-test or one-way ANOVA followed by a post hoc t-test with Bonferroni correction as appropriate. For some studies, results were plotted vs duration after start of experiments and the slope of the lines were calculated by the least mean squares method. For statistical analysis, results for groups of pigs given LPS+/−PIP-2 were compared with each other and with baseline pigs, i.e., pigs that were not subjected to mechanical ventilation or given LPS; control pigs, i.e. mechanically ventilated but no LPS, were not included in the statistical evaluation because of insufficient numbers (n=2). Statistical significance was assessed with SigmaStat software (Jandel Scientific, San Jose, CA). Statistical significance for all studies was accepted as p<0.05.

### Results

### Overview

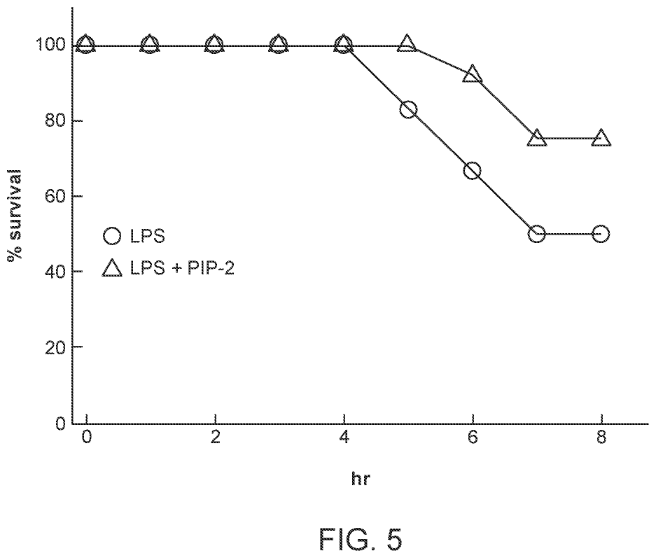

Control pigs that were untreated (no LPS, n=2) showed only minor changes in cardiovascular and pulmonary parameters vs. baseline measurements, indicating that 8 hours of mechanical ventilation under anesthesia was well tolerated (see data below). Based on humane considerations as described above, 50% (3 of 6) of pigs given LPS alone were sacrificed prior to the 8 hour time point planned for study while only 25% (3 of 12) of pigs given LPS and treated with PIP-2 were sacrificed for similar considerations (FIG. 5).

### Pulmonary Parameters

The aPO₂ was measured at hourly intervals and used to calculate the arterial blood PO₂ divided by the fractional inspired O₂ (FIO₂); this parameter (aPO₂/FIO₂) has been called the Horowitz index. Since the fractional O₂ used in this study for ventilation was 1.0, the Horowitz index in these studies is identical to the aPO₂. At both 4 and 8 hours of assisted ventilation, the aPO₂ (Horowitz index) of the 2 control animals was unchanged from baseline (Table 9). The aPO₂ in the LPS treated pigs (group 2) was decreased significantly ~65% at 4 hours (n=6) and about the same at 8 hours (n=3) after the start of LPS infusion (Table 9). By contrast, the mean aPO₂ was decreased only 50% at 4 hours (n=12) and 37% at 8 hours (n=9) after the start of LPS infusion (Table 9).

TABLE 9
Arterial Blood PO2 in pigs at baseline and
at 4 and 8 hrs after start of LPS infusion

| | Condition | |
|---|---|---|
| | Arterial Blood PO₂ (aPO₂) mmHg | |
| | Base line (n = 20) | |
| | 574 ± 60 | |
| | 4 h | 8 h |
| Control (group 1) | 578 ± 54 (n = 2) | 549 ± 107 (n = 2) |
| LPS (group 2) | 187 ± 119* (n = 6) | 193 ± 117* (n = 3) |
| LPS + PIP-2 (group 3) | 287 ± 184* (n = 12) | 362 ± 173* (n = 9) |

The arterial blood PO2 was measured while animals were ventilated with 100% O2 . . . N = 20 for baseline; N = 2 at 4 and 8 hrs for Control; N = 6 and 3 for LPS at 4 hr and at 8 hrs, respectively; N = 12 and 9 for LPS + PIP-2 at 4 and 8 hrs, respectively. Results are mean + SD or mean + range for n = 2.
*P < 0.05 compared to baseline.

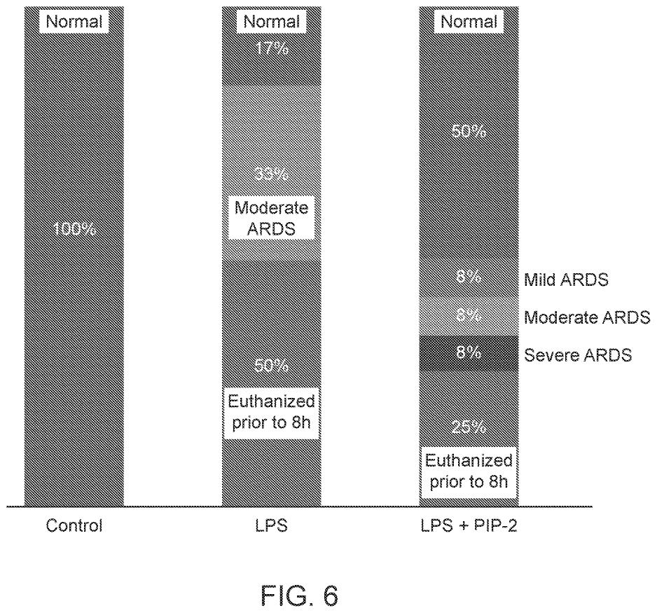

The aPO₂/FiO₂ (Horowitz index) has been the major parameter used to grade the severity of ALI. When evaluated at the end of the 8 hour study period, moderate to severe ALI was noted in 2 of the remaining 3 (67%) untreated LPS pigs but in only 2 of the 9 remaining PIP-2 treated pigs (22%) (FIG. 6). Thus, either normal lungs or mild ARDS was observed in 58% of PIP-2 treated but in only 17% of pigs that did not receive PIP-2. Consequently, PIP-2 treatment resulted in a 50% decrease in pig mortality by humane sacrifice and a 67% decrease in the incidence of moderate to severe ARDS in the pigs that survived for the entire 8 hour study duration. At 8 hours after the start of LPS infusion, 50% of the PIP-2 treated pigs (Group 3) but only 17% of LPS alone pigs (Group 2) had aPO₂ within the normal range (FIG. 2).

### Blood Assays

All pigs in Groups 2 (LPS+liposomes) and 3 (LPS+PIP-2 in liposomes) developed lactic acidosis within 30 to 60 minutes after the initiation of LPS infusion. Lactate levels stabilized but remained slightly elevated for both groups over the study duration with essentially no difference between the 2 groups. Similarly, there was no significant difference between PIP-2-treated and untreated pigs for measurements of blood BUN, creatinine, alkaline phosphatase, and aspartate amino transferase. On the other hand, the mean C-reactive protein, an indicator of systemic inflammation, was increased nearly 5-fold by administration of LPS and that increase was totally suppressed (p<0.05) in pigs treated with PIP-2 (Table 10). Thus, PIP-2 treatment of LPS-induced sepsis resulted in markedly lower blood C-reactive protein compared to untreated pigs.

TABLE 11
Values for blood analysis in pigs at baseline and
at 4 and 8 hrs after LPS +/− PIP-2.

| | Control | | | | | |
|---|---|---|---|---|---|---|
| | C-reactive | | White cell count | | Platelet count | |
| | protein μg/mL | | 10³/μl | | 10³/μL | |
| | Baseline (n = 20) | | | | | |
| | 63.9 ± 48 | | 14.6 ± 2.8 | | 394 ± 85 | |
| | 4 h | 8 h | 4 h | 8 h | 4 h | 8 h |
| Control | 23.0 ± | 59.3 ± | 21.3 ± | 21.4 ± | 418 ± | 403 ± |
| (group 1) | 34.4 | 24.5 | 4.7 | 4.2 | 154 | 94 |
| LPS | 167 ± | 277 ± | 1.81 ± | 5.7 ± | 158 ± | 146 ± |
| (group 2) | 131 | 57.3* | 0.5* | 2.6* | 67* | 69* |
| LPS + PIP-2 | 41.2 ± | 57.3 ± | 2.65 ± | 8.4 ± | 160 ± | 125 ± |
| (group 3) | 22.9 | 31.0 ^{ł} | 0.6* | 1.9* | 62* | 53* |

N = 20 for baseline; N = 2 at 4 and 8 hrs for Control; N = 6 and 3 for LPS at 4 hr and at 8 hrs, respectively; N = 12 and 9 for LPS + PIP-2 at 4 and 8 hrs, respectively. Results are mean + SD or mean + range for n = 2.
*P < 0.05 compared to baseline;
^{ł} P < 0.05 compared to LPS.

Pigs given LPS showed significant depression of the blood white cell (WBC) and platelet counts (Table 10) compatible with the response to sepsis. The mean PMN leukocyte count after LPS (group 2) was decreased by ~80-90% vs control at both 4 and 8 hours but by only ~60% and 40% at 4 and 8 hours, respectively, in animals treated with PIP-2 (Table 11), although these differences between plus or minus PIP-2 were not statistically significant. PIP-2 treatment had no effect on the decrease in blood platelets associated with LPS.

### Cardiovascular Parameters

Group 2 (LPS) animals developed elevation in the plasma levels of cardiac troponin and of creatine kinase at the 4 and 8 hour observation periods (Table 12). These enzymes are considered to be indicators of cardiac muscle injury. There was no change from baseline in levels of these 2 enzymes in the group 3 (LPS+PIP-2) animals (Table 12). Thus, the increases in serum troponin and creatine kinase in the group 2 pigs were statistically significant as compared to the group 3 (PIP-2) treated pigs.

TABLE 12
Values for cardiovascular-related enzymes in pigs at baseline
and at 4 and 8 hrs after the start of LPS infusion.

| | Condition | | | |
|---|---|---|---|---|
| | Troponin ng/mL | | Creatine Kinase U/mL | |
| | Base line | | | |
| | 0.06 ± 0.03 | | 359 ± 164 | |
| | 4 h | 8 h | 4 h | 8 h |
| Control | 0.08 ± 0.05 | 0.07 ± 0.04 | 181 ± 1 | 314 ± 118 |
| (group 1) | | | | |
| LPS | 0.23 ± 0.25* | 0.90 ± 0.94 | 430 ± 130 | 984 ± 402* |
| (group 2) | | | | |
| LPS + PIP-2 | 0.08 ± 0.07 | 0.10 ± 0.08 | 364 ± 87 | 529 ± 180 |
| (group 3) | | | | |

N = 20 for baseline; N = 2 at 4 and 8 hrs for Control; N = 6 and 3 for LPS at 4 hr and at 8 hrs, respectively; N = 12 and 9 for LPS + PIP-2 at 4 and 8 hrs, respectively. Results are mean + SD or mean + range for n = 2.
*P < 0.05 compared to baseline.

Considerable cardiovascular instability was noted in LPS-treated pigs with a significant increase in heart rate and decreased arterial blood pressure (Table 13). The latter required the administration of vasopressors (i.e., norepinephrine, epinephrine, and/or phenylephrine) in order to maintain systemic arterial blood pressure within the normal range. Substantially less (80-90%) supportive therapy was required to maintain acceptable arterial blood pressure values in pigs treated with PIP-2 (group 3) compared to untreated pigs (group 2) (Table 13).

TABLE 13
Cardiovascular measurements in pigs at baseline
and at 4 and 8 hrs after start of LPS infusion.

| | Condition | | | | | |
|---|---|---|---|---|---|---|
| | Heart rate | | Mean Arterial Blood | | Total pressor | |
| | beats/min | | Pressure, mmHg | | use mg | |
| | Baseline | | | | | |
| | 85 ± 10 | | 87 ± 11 | | 0.0 ± 0.0 | |
| | 4 h | 8 h | 4 h | 8 h | 4 h | 8 h |
| Control | 95 ± | 96 ± | 92 ± | 118 ± | 0.0 ± | 0.0 ± |
| (group 1) | 14 | 42 | 0 | 1 | 0.0 | 0.0 |
| LPS | 182 ± | 144 ± | 63 ± | 87 ± | 8.90 ± | 8.61 ± |
| (group 2) | 33* | 41* | 12 | 21 | 6.49 | 6.33* |
| LPS + PIP-2 | 163 ± | 132 ± | 71 ± | 93 ± | 3.03 ± | 1.06 ± |
| (group 3) | 41 | 12 | 12 | 23 | 3.61 | 0.87 |

Total pressor is the total amount of epinephrine, norepinephrine, and phenylephrine given to maintain adequate arterial blood pressure during the initial 4 hrs and during the subsequent 4-8 hrs of the experiment. N = 20 for baseline; N = 2 at 4 and 8 hrs for Control; N = 6 and 3 for LPS at 4 hr and at 8 hrs, respectively; N = 12 and 9 for LPS + PIP-2 at 4 and 8 hrs, respectively. Results are mean + SD or mean + range for n = 2.
*P < 0.05 vs

baseline.

### Pulmonary Parameters—Lung Compliance

In addition to measurements of PaO₂ that were compatible with ALI, lung injury was assessed by measurement of lung compliance and analysis of broncho-pulmonary lavage fluid. Decreased lung compliance indicating stiff lungs is a common feature of ALI. The mean value for static lung compliance (CL) was reduced from 20.3 ml/cm H₂O for pigs at baseline to 11.0 ml/cm H₂O (436% decrease) for group 2 pigs at 4 hours after the start of LPS infusion, with essentially no further change in the mean value at 8 hours of study (Table 14). Treatment of pigs with PIP-2 (group 3) resulted in a lesser decrease (40% at 4 hours and 30% at 8 hours) in lung compliance compared with untreated (group 2, no PIP-2) pigs. For both Group 2 and group 3 pigs, the major decrease in lung compliance occurred during the initial 4 hours of the experiment.

TABLE 14
Static lung compliance of pigs at baseline and
at 4 and 8 hrs after start of LPS infusion.

Condition
Static Lung Compliance mL/cm H₂O
Base line
20.3 ± 4

| | 4 h | 8 h |
|---|---|---|
| Control (group 1) | 20.6 ± 3.0 | 17.3 ± 5.2 |
| LPS (group 2) | 11.0 ± 2.0* | 11.2 ± 3.8* |
| LPS + PIP-2 (group 3) | 12.2 ± 4.0* | 14.1 ± 3.6* |

N = 20 for baseline; N = 2 at 4 and 8 hrs for Control; N = 6 and 3 for LPS at 4 hr and at 8 hrs, respectively; N = 12 and 9 for LPS + PIP-2 at 4 and 8 hrs, respectively. Results are mean + SD or mean + range for n = 2.
*P < 0.05 compared to baseline.

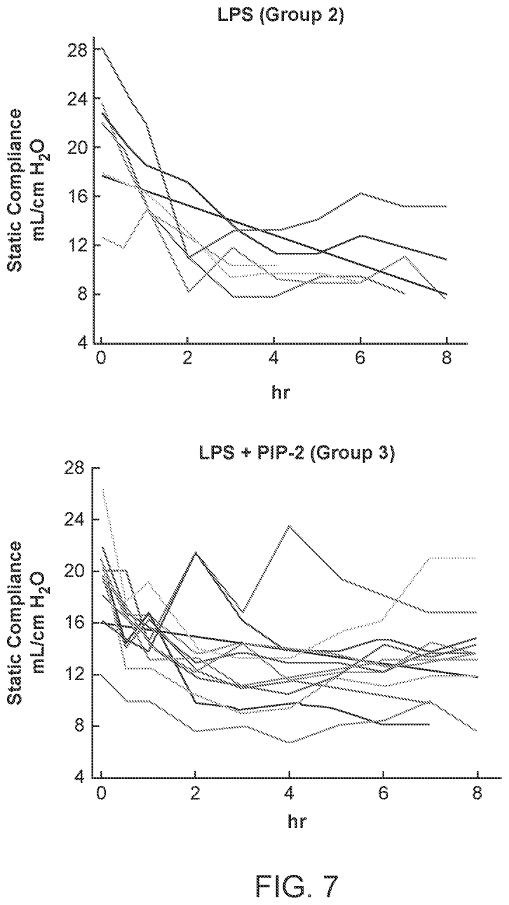

The lung compliance data was analyzed further by plotting values obtained at hourly intervals for pigs in group 2 and in group 3 (FIG. 7). The linear slopes under the 2 conditions indicated a statistically significant lesser decrease in lung compliance (P<0.001) in pigs treated with PIP-2.

### Pulmonary Parameters, Broncho-Alveolar Lavage:

Twenty pigs were subjected to lung lavage at the end of the experiment; the lavage fluid from one pig was lost and not analyzed. The fluid from the 19 pigs that were analyzed included: Control (n=2), LPS (n=5), and LPS+PIP-2 (n=12) groups. For the LPS group, 3 of the lavages were done at the 8 hour termination point of experiments and 2 were done with pigs subjected to early euthanasia between 5 and 6 hours after start of LPS infusion. For the LPS+PIP-2 group, 3 pigs were subjected to early euthanasia and then lavaged but all 3 yielded bloody lavage fluid and these samples were discarded without further analysis. Thus, analysis of lavage fluid was recorded for BALF from 16 pigs.

Mean total protein in BALF was increased significantly (~3.5-fold) in pigs administered LPS alone (group 2); the increase in protein was 50% less with administration of PIP-2 (group 3) (Table 15) indicating a more intact alveolar-capillary barrier. Lung inflammation was evaluated by the expression of MPO and cytokines. Despite the systemic leukopenia, there was a nearly 6-fold increase in myeloperoxidase (MPO) content in lung lavage fluid from pigs given LPS alone (Table 15); MPO is generally accepted as reflecting the presence of PMNs. The increase in MPO with LPS was abolished by treatment with PIP-2. Likewise, lung lavage cytokines (IL-6, IL-1β, TNFα) were increased 2-3 fold with LPS treatment and this increase was blocked by treatment with PIP-2 (Table 15). Thus, PIP-2 treatment of LPS-exposed animals had a protective effect on lung alveolar capillary protein permeability and decreased lung inflammation as indicated by BALF MPO and cytokine levels.

TABLE 15
Analysis of broncho-alveolar lavage fluid (BALF).

| | Lavage prot. | IL-6 pg/kg | IL-1β pg/ | TNF-α pg/ | MPO ng/kg |
|---|---|---|---|---|---|
| Condition | mg/kg body wt | body wt. | kg body wt. | kg body wt. | body wt. |
| Control (group 1) | 2.27 ± 1.35 | 596 ± 339 | 257 ± 203 | 203 ± 83 | 10.2 ± 6.4 |
| LPS (group 2) | 4.70 ± 0.29 | 2330 ± 141 | 1120 ± 233 | 902 ± 5 | 205 ± 8 |
| | 5.17 ± 0.63 | 3000 ± 61 | 1230 ± 104 | 1043 ± 70 | 248 ± 23 |
| LPS + PIP-2 (group 3) | 1.39 ± 0.46* | 390 ± 127* | 183 ± 54* | 144 ± 58* | 1.4 ± 0.5* |

For groups 1 (n = 2) and 3 (n = 9), BALF studies were done in pigs sacrificed at 8 hr after start of LPS infusion. For group 2, BALF studies were done for pigs sacrificed at 5-6 hrs (n = 3) and at 8 hrs (n = 3). The values for group 2 pigs sacrificed at 5-6 hrs is shown on the upper line, and the values for pigs sacrificed at 8 hrs are shown on the lower line. Results are mean + SD or mean + range for n = 2.
*P < 0.05 for LPS + PIP-2 (group 2) at both 5-6 hrs and at 8 hrs; none of the other comparisons are statistically significant (P > 0.05).

### Histology

The control animals showed relatively trivial evidence of lung injury compatible with tolerance to mechanical ventilation for 8 hours under control conditions. Lungs from LPS-treated animals demonstrated evidence of mild to moderate inflammation with no significant difference between PIP-2 treated and PIP-2 untreated pigs. Of note, the lungs were not inflation-fixed, which limited the utility of the morphologic examination. Examination of heart, liver and kidneys likewise showed relatively minor inflammation and hemorrhage that was not significantly different between the PIP-2 treated and untreated animals. The relatively minor changes at post-mortem examination presumably reflect the relatively short duration (<8 hours) of LPS exposure.

### Summary of Results

To summarize the cardiovascular-pulmonary results shown in Tables 9-Table 15 and FIGS. 4-FIG. 7, treatment with PIP-2 resulted in: 1) a 50% decrease in mortality; 2) a 3-fold increase in pigs that maintained a normal arterial PO2; 3) prevented elevations of serum troponin and creatine kinase following LPS compatible with decreased cardiac muscle injury; 4) markedly decreased the requirement for vasopressor drugs to maintain arterial blood pressure; 5) resulted in a significant decrease in lung stiffness (improved lung compliance); 6) decreased the LPS-mediated change in alveolar-capillary protein permeability; and 7) decreased lung inflammation as indicated by both decreased cytokine release and decreased MPO content in lung lavage fluid.

### DISCUSSION

A major feature of ALI is lung inflammation and the release of potentially damaging agents from inflammatory cells. These damaging agents include reactive oxygen species (ROS) that, when produced in excess, can oxidize tissue macromolecules resulting in widespread cellular destruction. ROS can be produced in the lung through several mechanisms, with a major source being the enzymatic activity of NADPH oxidase (NOX) enzymes, essentially NOXes1 and 2. An extensive recent review described how oxidant stress plays an important role in many acute and chronic disease states (Forman H J, Zhang H. Targeting oxidative stress in disease: promise and limitations of antioxidant therapy. Nat Rev Drug Discov. 2021 September; 20(9):689-709. doi: 10.1038/s41573-021-00233-1. Epub 2021 Jun. 30. PMID: 34194012). That review states that small peptides that inhibit the assembly of the NOX oxidases have therapeutic potential but points out that none of the proposed inhibitors has advanced to clinical trials. Although ALI/ARDS was not discussed specifically in the review, clearly excess ROS production resulting in oxidant stress plays an important role in lung injury with this syndrome.

The study presented herein utilized a small (9 amino acid) peptide that inhibited the activation of NOXes 1 and 2 by inhibiting rac release from the cell membrane, thus inhibiting assembly of the oxidase. Inhibition of rac release results from inhibition of the aiPLA2 activity of Prdx6. This role for aiPLA2 activity in the activation of NOXes 1 and 2 was demonstrated by use of either MJ33 or PIP-2 to inhibit the aiPLA2 activity of Prdx6 and also by mutation of the Prdx6 molecular sites required for expression of aiPLA2 activity. Because MJ33 is a lipid molecule that inhibits various PLA2 enzymes such as pancreatic PLA2 as well as the aiPLA2 activity of Prdx6, inhibition of NOX2 activation was demonstrated to be associated with inhibition of aiPLA2 (the PLA2 activity of Prdx6). The surfactant-associated protein SP-A binds to Prdx6 and inhibits its aiPLA2 activity. Protein truncation was used to determine the minimal effective sequence of the 246 amino acids of human SP-A to inhibit aiPLA2. A 9 amino acid sequence, called phospholipase A2 inhibitory peptide (PIP), was determined to have full inhibitory activity. The PIP sequence for human SP-A was called PIP-2.

Mouse studies using several different models of ALI have shown that inhibition of rac 1,2 activation by either MJ33 or PIP-2 had a markedly protective effect against lung injury. Mouse models of ALI showed protection against lung injury by treatment with one of the rac inhibitors. The study presented herein showed that inhibition of ROS production through inhibition of NOX2 in LPS-induced sepsis plus ALI also was protective against manifestations of pulmonary and cardiovascular injury in pigs, an animal species with lungs that more closely resemble the anatomy and physiology of human lungs.

As evidence for protection by PIP-2, the mortality rate (based on IACUC-directed humane sacrificed animals) was decreased by 50% in PIP-2 treated pigs. Secondly, 50% of PIP-2-treated, but only 17% of untreated pigs, maintained normal arterial blood oxygenation for the duration of the experiment. Additional observed beneficial effects of PIP-2 treatment included: a significant reduction in the increase of indicators of injury to heart muscles as indicated by serum troponin 1 and creatine kinase levels; a significant reduction in the pressor requirements for arterial blood pressure control; a reduced alteration of lung gas exchange as indicated by a decreased abnormal alveolar to arterial (A-a) PO₂ gradient; a protection of the lung alveolar-capillary barrier as indicated by a significant reduction in the leakage of protein into the lung alveolar space; a lesser decrease in blood WBC compatible with a decrease in the manifestations of sepsis; a significant reduction in the elevated blood C-reactive protein also compatible with lessened sepsis; and decreased lung inflammation as indicated by a decrease in lung lavage WBC (estimated by MPO content). Of note, for all parameters studied, treatment with PIP-2 either had no effect on the manifestations of LPS-induced injury and sepsis or provided a positive effect; that is, none of the parameters measured were significantly worsened by treatment with PIP-2. Thus, use of PIP-2 was protective in this model of ALI with no evidence of adverse side-effects.

The effectiveness of PIP-2 as potential agent for the treatment of clinical ALI and/or sepsis is based on the important role for NOX-generated ROS in the pathophysiology of lung injury. Excessive ROS generation is considered to be of major importance in the pathophysiology of acute lung injury and inhibition of NOX1/2-generated ROS is expected to significantly reduce the oxidative stress associated with this syndrome. Thus, PIP-2 as a non-toxic inhibitor with a relatively favorable tissue half-life can be considered as a strong candidate for testing as adjunctive therapy for prevention or treatment of sepsis and/or ALI.

In sum, PIP-2 reduce mortality in animals in septic shock by 50%, a result which has not been observed in other studies in this model. In addition, biomarkers for PIP-2 showed near normal levels in the treated animals, while the placebo animals showed significant injury. Without wishing to be bound by theory, due to the nature of sepsis, the results suggest that the placebo animals would have additional mortality due to the poor biomarkers observed. However, some of the mild and modestly affected animals in the PIP-2 treatment group may fully recover, as their biomarkers showed near normal levels after PIP-2 treatment.

In addition, this study demonstrated several unexpected benefits of PIP-2 treatment. First, troponin levels of the placebo arm showed the expected rapid increase observed septic shock injury. However the PIP-2 treatment animals showed near normal troponin levels. Without wishing to be bound by theory, PIP-2 treatment provided cardio protection. PIP-2's ability to protect cardio function in these studies was unexpected. No other compounds tested to date demonstrated the cardio protective effect so quickly and significantly. Secondly, use of vasopressors is a standard of care in septic shock injury. One of the impact in septic shock is the loss of perfusion due to endothelial dysfunction and the most common treatment is the use of vasopressors. Loss of perfusion is a common cause of death in septic shock injuries. In this study, the PIP-2 treated animals required a third (⅓) less total mg of vasopressor than the placebo animals, while also achieving greater mean arterial pressure. Achieving this type of mean arterial pressure in a septic shock injury with a novel mechanism of was also unexpected and can have a beneficial effect in patients, since the use of vasopressors have many negative side effects that PIP-2 did not demonstrate. Therefore, the use of PIP-2 and the related peptides provide significant advantages over the current standard of care for septic shock.

This specification contains numerous citations to patents, patent applications, accession numbers, and/or publications. Each is hereby incorporated by reference for all purposes.

What is claimed is:

1. A method of treating septic shock in a subject in need thereof, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
wherein:
X¹ is E or is absent;
X² is L or is absent;
X³ is Q or is absent;
X⁴ is A, T, or is absent;
X⁵ is T, E, or is absent;
X⁶ is H or Y;
X⁷ is D or E;
X⁸ is F or I; and
X⁹ is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier.

2. The method of claim 1, wherein the polypeptide comprises an amino acid sequence that is at least 85%, 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

3. The method of claim 1, wherein the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

4. The method of claim 1, wherein the polypeptide comprises SEQ ID NO: 2.

5. The method of claim 1, wherein the polypeptide consists of SEQ ID NO: 2.

6. The method of claim 1, wherein the polypeptide comprises SEQ ID NO: 3.

7. The method of claim 1, wherein the polypeptide consists of SEQ ID NO: 3.

8. The method of claim 1, wherein the polypeptide comprises SEQ ID NO: 4.

9. The method of claim 1, wherein the polypeptide consists of SEQ ID NO: 4.

10. The method of claim 1, wherein the pharmaceutical composition comprises a liposome encapsulated polypeptide.

11. The method of claim 1, wherein the pharmaceutically acceptable carrier is any pharmaceutically acceptable carrier disclosed herein.

12. The method of claim 1, wherein the pharmaceutical composition is administered to the subject by aerosol inhalation, intratracheal injection, or intravenous injection.

13. The method of claim 1, wherein the subject in need thereof has been diagnosed with sepsis and has low blood pressure that is not alleviated by the administration of intravenous fluids alone.

14. The method of claim 13, wherein the subject in need thereof has a mean arterial pressure (MAP) less than about 65 mm Hg.

15. The method of claim 1, wherein the subject in need thereof has been diagnosed with sepsis and has a decrease in mean arterial pressure or systolic blood pressure of about 44 mm Hg or more.

16. The method of claim 1, wherein the subject in need thereof has blood lactate levels that are greater than about 4 mmol.

17. The method of claim 1, wherein the subject in need thereof has persistent signs of multiple organ damage or failure.

18. The method of claim 1, wherein the subject's blood oxygen level does not decrease after administration of the pharmaceutical composition.

19. The method of claim 18, wherein the subject's blood oxygen level does not decrease more than 50% or 65% after administration of the pharmaceutical composition.

20. The method of claim 1, wherein the subject's blood oxygen level increases after administration of the pharmaceutical composition.

21. The method of claim 1, wherein the subject maintains a normal blood oxygen level after administration of the pharmaceutical composition.

22. The method of claim 1, wherein the subject in need thereof is not administered a vasopressor or the subject has been previously administered a lower amount of a vasopressor, is concurrently administered a lower amount of a vasopressor, or will subsequently be administered a lower amount of a vasopressor than the amount of a vasopressor administered in the absence of the pharmaceutical composition.

23. A method of treating subject with a significant drop in mean arterial pressure, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
wherein:
X¹ is E or is absent;
X² is L or is absent;
X³ is Q or is absent;
X⁴ is A, T, or is absent;
X⁵ is T, E, or is absent;
X⁶ is H or Y;
X⁷ is D or E;
X⁸ is F or I; and
X⁹ is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier.

24. The method of claim 23, wherein the polypeptide comprises an amino acid sequence that is at least 85%, 90%, 91%, 92%, 93%, 94%, 95%, 96%, 97%, 98%, or 99% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

25. The method of claim 23, wherein the polypeptide comprises an amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

26. The method of claim 23, wherein the polypeptide comprises SEQ ID NO: 2.

27. The method of claim 23, wherein the polypeptide consists of SEQ ID NO: 2.

28. The method of claim 23, wherein the polypeptide comprises SEQ ID NO: 3.

29. The method of claim 23, wherein the polypeptide consists of SEQ ID NO: 3.

30. The method of claim 23, wherein the polypeptide comprises SEQ ID NO: 4.

31. The method of claim 23, wherein the polypeptide consists of SEQ ID NO: 4.

32. The method of claim 23, wherein the pharmaceutical composition comprises a liposome encapsulated polypeptide.

33. The method of claim 23, wherein the pharmaceutically acceptable carrier is any pharmaceutically acceptable carrier disclosed herein.

34. The method of claim 23, wherein the pharmaceutical composition is administered to the subject by aerosol inhalation, intratracheal injection, or intravenous injection.

35. The method of claim 23, wherein the subject has a decrease in mean arterial pressure or systolic blood pressure of about 44 mm Hg or more and has been diagnosed with sepsis.

36. The method of claim 23, wherein the subject has been diagnosed with sepsis and has low blood pressure that is not alleviated by the administration of intravenous fluids alone.

37. The method of claim 36, wherein the subject has a mean arterial pressure (MAP) less than about 65 mm Hg.

38. The method of claim 23, wherein the subject in need thereof has blood lactate levels that are greater than about 4 mmol.

39. The method of claim 23, wherein the subject in need thereof has persistent signs of multiple organ damage or failure.

40. The method of claim 23, wherein the subject's blood oxygen level does not decrease after administration of the pharmaceutical composition.

41. The method of claim 40, wherein the subject's blood oxygen level does not decrease more than 50% or 65% after administration of the pharmaceutical composition.

42. The method of claim 23, wherein the subject's blood oxygen level increases after administration of the pharmaceutical composition.

43. The method of claim 23, wherein the subject maintains a normal blood oxygen level after administration of the pharmaceutical composition.

44. The method of claim 23, wherein the subject in need thereof is not administered a vasopressor or the subject has been previously administered, is concurrently administered, or will subsequently be administered a lower amount of a vasopressor than the amount of a vasopressor administered in the absence of the pharmaceutical composition.

45. The method of claim 23, wherein the subject in need thereof has been diagnosed with septic shock.

46. A method of preventing heart damage in a subject with diagnosed with septic shock, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
wherein:
X¹ is E or is absent;
X² is L or is absent;
X³ is Q or is absent;
X⁴ is A, T, or is absent;
X⁵ is T, E, or is absent;
X⁶ is H or Y;
X⁷ is D or E;
X⁸ is F or I; and
X⁹ is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier, as compared to a subject not treated with the pharmaceutical composition.

47. The method of claim 46, wherein the polypeptide comprises an amino acid sequence that is at least 85%, 90%, 91%, 92%, 93%, 94%, 95%, 96%, 97%, 98%, 99% or 100% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.

48. A method of increasing survival of a subject with diagnosed with septic shock, the method comprising administering to the subject an effective amount of a pharmaceutical composition comprising a polypeptide comprising an amino acid sequence of:
X¹X²X³X⁴X⁵LX⁶X⁷X⁸X⁹HQIL (SEQ ID NO: 1)
wherein:
X¹ is E or is absent;
X² is L or is absent;
X³ is Q or is absent;
X⁴ is A, T, or is absent;
X⁵ is T, E, or is absent;
X⁶ is H or Y;
X⁷ is D or E;
X⁸ is F or I; and
X⁹ is R or K; or
an amino acid sequence selected from the group consisting of SEQ ID NOs: 10, 11, 12, 13, 14, 15, 17, 18, 19, 20, 21, 22, 24, 25, 26, 27, 28, 29, or 30;
and a pharmaceutically acceptable carrier, as compared to a subject not treated with the pharmaceutical composition.

49. The method of claim 48, wherein the polypeptide comprises an amino acid sequence that is at least 85%, 90%, 91%, 92%, 93%, 94%, 95%, 96%, 97%, 98%, 99% or 100% identical to amino acid sequence selected from the group consisting of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, and 30.


SEQUENCE LISTING (30 sequences)

SEQ ID NO: 1 - protein, 14 aa, synthetic construct
  VARIANT 1: note = X can be E or absent
  VARIANT 2: note = X can be L or absent
  VARIANT 3: note = X can be Q or absent
  VARIANT 4: note = X can be A, T or absent
  VARIANT 5: note = X can be T, E or absent
  VARIANT 7: note = X can be H or Y
  VARIANT 8: note = X can be D or E
  VARIANT 9: note = X can be F or I
  VARIANT 10: note = X can be R or K
XXXXXLXXXXHQIL

SEQ ID NO: 2 - protein, 9 aa, synthetic construct
LHDFRHQIL

SEQ ID NO: 3 - protein, 9 aa, synthetic construct
LYEIKHQIL

SEQ ID NO: 4 - protein, 9 aa, synthetic construct
LYDIRHQIL

SEQ ID NO: 5 - protein, 16 aa, synthetic construct
DEELQATLHDFRHQIL

SEQ ID NO: 6 - protein, 16 aa, synthetic construct
DEELQTELYEIKHQIL

SEQ ID NO: 7 - protein, 14 aa, synthetic construct
ELQTELYEIKHQIL

SEQ ID NO: 8 - protein, 12 aa, synthetic construct
QTELYEIKHQIL

SEQ ID NO: 9 - protein, 10 aa, synthetic construct
ELYEIKHQIL

SEQ ID NO: 10 - protein, 8 aa, synthetic construct
YEIKHQIL

SEQ ID NO: 11 - protein, 6 aa, synthetic construct
IKHQIL

SEQ ID NO: 12 - protein, 14 aa, synthetic construct
DEELQTELYEIKHQ

SEQ ID NO: 13 - protein, 12 aa, synthetic construct
DEELQTELYEIK

SEQ ID NO: 14 - protein, 11 aa, synthetic construct
DEELQTELYEI

SEQ ID NO: 15 - protein, 8 aa, synthetic construct
DEELQTEL

SEQ ID NO: 16 - protein, 10 aa, synthetic construct
TLHDFRHQIL

SEQ ID NO: 17 - protein, 9 aa, synthetic construct
TLHDFRHQI

SEQ ID NO: 18 - protein, 8 aa, synthetic construct
LHDFRHQI

SEQ ID NO: 19 - protein, 9 aa, synthetic construct
ELYEIKHQI

SEQ ID NO: 20 - protein, 8 aa, synthetic construct
LYEIKHQI

SEQ ID NO: 21 - protein, 9 aa, synthetic construct
LKIEYHQIL

SEQ ID NO: 22 - protein, 9 aa, synthetic construct
LRFDHHQIL

SEQ ID NO: 23 - protein, 9 aa, synthetic construct
LHEFKHQIL

SEQ ID NO: 24 - protein, 9 aa, synthetic construct
LFKLEHQIL

SEQ ID NO: 25 - protein, 9 aa, synthetic construct
LHDFRDQIL

SEQ ID NO: 26 - protein, 9 aa, synthetic construct
LHDFRPQIL

SEQ ID NO: 27 - protein, 9 aa, synthetic construct
LHDFRHNIL

SEQ ID NO: 28 - protein, 9 aa, synthetic construct
LHDFRHIIL

SEQ ID NO: 29 - protein, 9 aa, synthetic construct
LHDFRHQLL

SEQ ID NO: 30 - protein, 9 aa, synthetic construct
LHDFRHQTL